(19) United States
(12) Patent Application Publication
(10) Pub. No.: US 2003/0027774 A1
(43) Pub. Date: Feb. 6, 2003

(54) TUBERCULOSIS ANTIGENS AND METHODS OF USE THEREFOR

(76) Inventors: RONALD C. HENDRICKSON, SEATTLE, WA (US); MICHAEL J. LODES, SEATTLE, WA (US); RAYMOND L. HOUGHTON, BOTHELL, WA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/272,975

(22) Filed: Mar. 18, 1999

(51) Int. Cl. A61K 31/70; A01N 43/04

(52) U.S. Cl. 514/44

(57) ABSTRACT

Compounds and methods for the diagnosis and treatment of tuberculosis are disclosed. Compounds include the M. tuberculosis antigens Mtb-81 and Mtb-67.2, immunogenic portions thereof and polynucleotides that encode such portions. Such compositions may be used, for example, for the immunotherapy and serodiagnosis of M. tuberculosis infection.

### TECHNICAL FIELD

The present invention relates generally to the detection and treatment of tuberculosis. The invention is more specifically related to polypeptides comprising at least a portion of a Mycobacterium tuberculosis antigen, or a portion or other variant thereof, and to the use of such polypeptides for the serodiagnosis and immunotherapy of M. tuberculosis infection.

### BACKGROUND OF THE INVENTION

Tuberculosis is a chronic, infectious disease that is generally caused by infection with Mycobacterium tuberculosis. It is a major disease in developing countries, as well as an increasing problem in developed areas of the world, with about eight million new cases and three million deaths each year. Although the infection may be asymptomatic for a considerable period of time, the disease is most commonly manifested as an acute inflammation of the lungs, resulting in fever and a nonproductive cough. If left untreated, M. tuberculosis infection generally results in serious complications and death.

Inhibiting the spread of tuberculosis requires accurate, early diagnosis of the disease. The most common method of diagnosis is a skin test, which involves intradermal exposure to tuberculin PPD (protein-purified derivative). Antigen-specific T cell responses result in measurable indubation at the injection site within 48-72 hours after injection, which indicates exposure to mycobacterial antigens. Although the tuberculin test is used throughout the world, it suffers from problems with sensitivity and specificity. For example, individuals vaccinated with Bacillus Calmette-Guerin (BCG) cannot be distinguished from infected individuals. In addition, tuberculosis is a frequent occurrence in AIDS patients, but the sensitivity of the tuberculin skin test is substantially reduced during HIV infection.

Accordingly, there is a need in the art for improved diagnostic methods for detecting tuberculosis infection, particularly in HIV-infected individuals. The present invention fulfills these needs and further provides other related advantages.

### SUMMARY OF THE INVENTION

Briefly stated, this invention provides compositions and methods for the detection and therapy of tuberculosis. In certain aspects, isolated polypeptides are disclosed that comprise an immunogenic portion of one or both of the M. tuberculosis antigens referred to herein as Mtb-81 or Mtb-67.2. Alternatively, such polypeptides may comprise a variant of either antigen that differs in one or more substitutions, deletions, additions and/or insertions such that the ability of the variant to react with antigen-specific antisera is not substantially diminished. Within certain embodiments, the polypeptide comprises an amino acid sequence recited in FIGS. 1A-1F (SEQ ID NO:2) or FIG. 5 (SEQ ID NO:5). Fusion proteins comprising such polypeptides in combination with a known M. tuberculosis antigen are also provided.

Polynucleotides that encode all or a portion of an Mtb-81 or Mtb-67.2 polypeptide are also provided, as are antisense polynucleotides that comprise at least 15 consecutive nucleotides complementary to a sequence recited in FIGS. 1A-1F (SEQ ID NO:1) or FIG. 4 (SEQ ID NO:4). Recombinant expressions vectors comprising such polynucleotides, and host cells transformed or transfected with such polynucleotides, are also provided.

Within further aspects, the present invention provides antibodies, and antigen-binding fragments thereof, that specifically bind to Mtb-81 or Mtb-67.2. Such antibodies may be polyclonal or monoclonal.

Within certain aspects, the present invention provides methods for determining the presence or absence of M. tuberculosis infection in a biological sample. Certain such methods comprise the steps of: (a) contacting a biological sample with a polypeptide as recited above or an antigen-presenting cell that expresses such a polypeptide; (b) detecting in the sample an amount of immunocomplexes formed between the polypeptide and antibodies in the biological sample; and (c) comparing the amount of polypeptide with a cut-off value. Biological samples include, but are not limited to, whole blood, serum, sputum, plasma, saliva, cerebrospinal fluid and urine.

Other methods comprise the steps of: (a) contacting a biological sample that comprises T cells with an isolated polypeptide as described above; (b) detecting in the sample an amount of T cells that specifically react with the polypeptide; and (c) comparing the amount of T cells detected to a cut-off value.

Still further methods comprise the steps of: (a) detecting in a biological sample an amount of mRNA encoding a polypeptide as described above; and (b) comparing the amount of polynucleotide detected to a cut-off value. Within certain embodiments, the amount of mRNA is detected via polymerase chain reaction using, for example, at least one oligonucleotide primer that hybridizes to a polynucleotide that encodes a polypeptide as recited above, or a complement of such a polynucleotide. Within other embodiments, the amount of mRNA is detected using a hybridization technique, employing an oligonucleotide probe that hybridizes to a polynucleotide that encodes a polypeptide as recited above, or a complement of such a polynucleotide.

Other such methods comprise the steps of: (a) contacting a biological sample with an antibody or antigen-binding fragment as described above and (b) detecting in the sample an amount of immunocomplexes formed between antibody or antigen-binding fragment thereof and proteins in the biological sample. Such immunocomplexes may be detected, for example, using an ELISA or competitive assay.

Within related aspects, the present invention provides methods for determining the presence or absence of M. tuberculosis infection in a patient. Such methods may generally be performed using any of the methods provided above for determining the presence or absence of M. tuberculosis infection in a biological sample, with the biological sample obtained from a patient.

Within related aspects, methods are provided for monitoring therapy for M. tuberculosis infection in a patient. Certain methods comprise the steps of: (a) contacting a biological sample obtained from a M. tuberculosis-infected patient at a first time point with an isolated polypeptide or antigen-presenting cell as described above; (b) detecting an amount of immunocomplexes formed between the polypeptide and antibodies in the biological sample that specifically bind to the polypeptide; (c) repeating steps (a) and (b) using a biological sample obtained at a second time point, wherein the second time point follows at least a portion of therapy for M. tuberculosis infection; and (d) comparing the amount of immunocomplexes detected in step (a) with the amount detected in step (c).

Within other aspects, method for monitoring M. tuberculosis therapy in a patient may comprise the steps of: (a) detecting, in a biological sample obtained from a M. tuberculosis-infected patient at a first time point, an amount of a mRNA encoding a polypeptide as described above; (b) detecting an amount of such mRNA in a biological sample obtained from the patient at a second time point, wherein the second time point follows at least a portion of a therapy for M. tuberculosis infection; and (c) comparing the amount of mRNA detected in step (a) to the amount detected in step (b).

Other such methods comprise the steps of: (a) contacting a biological sample obtained from a M. tuberculosis-infected patient at a first time point with an antibody or antigen-binding fragment as described above; (b) detecting in the sample an amount of immunocomplexes formed between the antibody or antigen-binding fragment and proteins in the biological sample; (c) repeating steps (a) and (b) using a biological sample obtained at a second time point, wherein the second time point follows at least a portion of therapy for M. tuberculosis infection; and (d) comparing the amount of immunocomplexes detected in step (a) with the amount detected in step (c).

Within any of the methods recited above, the patient may be infected with HIV.

Within further aspects, diagnostic kits are provided. Such kits generally comprise a polypeptide, polynucleotide or antibody as described above. In addition, such kits may comprise a detection reagent or solid support material for use within the assays provided herein.

The present invention further provides, within other aspects, pharmaceutical compositions comprising: (a) a Mtb-81 or Mtb-67.2 polypeptide as described above; a polynucleotide encoding such a polypeptide; an antigen-presenting cell that expresses such a polypeptide; or an antibody or antigen-binding fragment thereof that specifically binds to Mtb-81 (SEQ ID NO:2) or Mtb-67.2 (SEQ ID NO:5); and (b) a physiologically acceptable carrier.

Within further aspects, the present invention provides vaccines comprising:(a) a Mtb-81 or Mtb-67.2 polypeptide as described above; a polynucleotide encoding such a polypeptide; or an antigen-presenting cell that expresses such a polypeptide; and (b) a non-specific immune response enhancer.

Methods are further provided, within other aspects, for inhibiting the development of tuberculosis in a patient, comprising administering to a patient an effective amount of (a) a polypeptide as described above, (b) a polynucleotide encoding such a polypeptide, (c) an antigen presenting cell that expresses a polypeptide or (d) an antibody or antigen-binding fragment thereof that specifically binds to Mtb-81 (SEQ ID NO:2) or Mtb-67.2 (SEQ ID NO:5), and thereby inhibiting the development of tuberculosis in the patient.

The present invention further provides methods for stimulating and/or expanding T cells specific for Mtb-81 or Mtb-67.2, comprising contacting T cells with one or more of: (i) a polypeptide as described above; (ii) a polynucleotide encoding such a polypeptide; and/or (iii) an antigen presenting cell that expresses such a polypeptide; under conditions and for a time sufficient to permit the stimulation and/or expansion of T cells. Isolated T cell populations prepared by such methods are also provided, as are methods for inhibiting the development of tuberculosis in a patient, comprising administering to a patient an effective amount of such a T cell population.

Within related aspects, the present invention provides methods for inhibiting the development of tuberculosis in a patient, comprising the steps of: (a) incubating CD4⁺ and/or CD8+ T cells isolated from a patient with one or more of: (i) a polypeptide as described above; (ii) a polynucleotide encoding such a polypeptide; or (iii) an antigen-presenting cell that expresses such a polypeptide; such that T cells proliferate; and (b) administering to the patient an effective amount of the proliferated T cells, and thereby inhibiting the development of tuberculosis in the patient.

Within further aspects, methods are provided for inhibiting the development of tuberculosis in a patient, comprising the steps of: (a) incubating CD4⁺ and/or CD8+ T cells isolated from a patient with one or more of: (i) a polypeptide as described above; (ii) a polynucleotide encoding such a polypeptide; or (iii) an antigen-presenting cell that expresses such a polypeptide; such that T cells proliferate; (b) cloning proliferated T cells; and (c) administering to the patient an effective amount of the proliferated T cells, and thereby inhibiting the development of tuberculosis in the patient.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

FIGS. 1A-1F depict a M. tuberculosis genomic sequence that includes a nucleotide sequence encoding Mtb-81. The predicted amino acid sequence of Mtb-81 is shown below the nucleotide sequence and is indicated by the solid black line.

FIG. 2 is a graph illustrating the seroreactivity of Mtb-81 in patients infected with HIV. Mtb-81 was used to detect reactive antibodies in sera from patients who were normal (uninfected with M. tuberculosis); HIV-positive and M. tuberculosis-positive; or HIV-negative and M. tuberculosis-positive, as indicated. OD₄₅₀ was indicative of antibody binding. Values above the cut-off value (indicated by the line) were considered positive for M. tuberculosis infection.

FIG. 3 is a graph illustrating the seroreactivity of Mtb-67.2 in tuberculosis patients co-infected with HIV. Mtb-67.2 was used to detect reactive antibodies in sera from patients who were normal (uninfected with M. tuberculosis); HIV-positive and M. tuberculosis-positive; or HIV-negative and M. tuberculosis-positive, as indicated. OD₄₅₀ was indicative of antibody binding. Values above the cut-off value (indicated by the line) were considered positive for M. tuberculosis infection.

FIG. 4 shows an M. tuberculosis DNA sequence encoding Mtb-67.2.

FIG. 5 shows an amino acid sequence of M. tuberculosis Mtb-67.2.

### DETAILED DESCRIPTION OF THE INVENTION

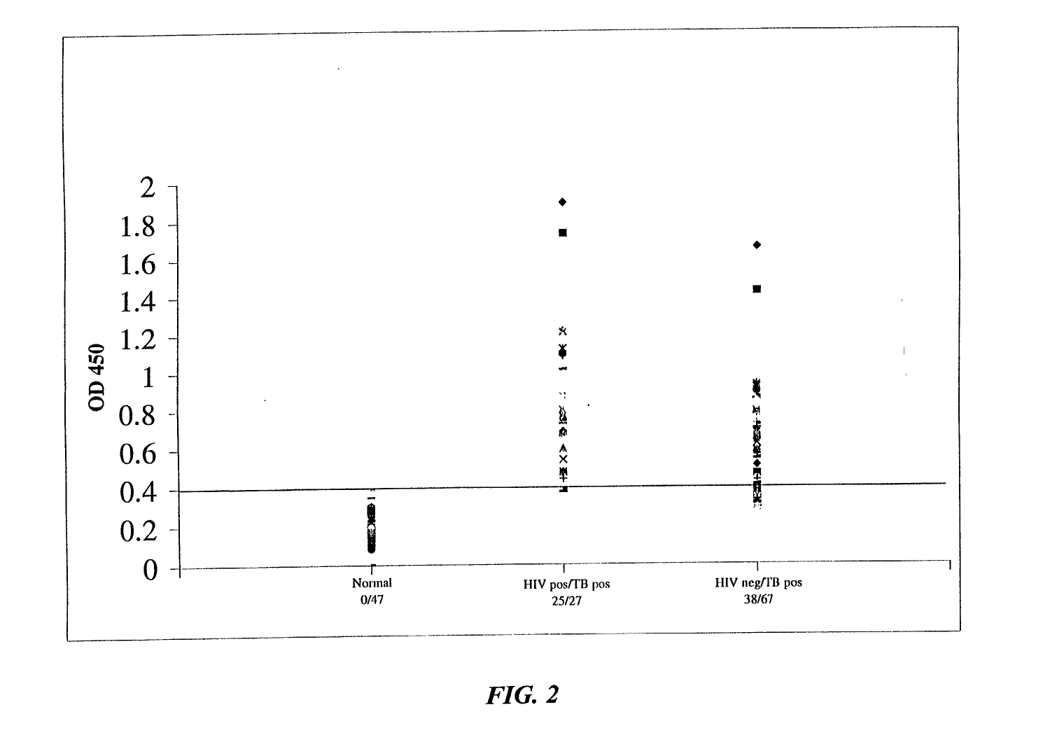

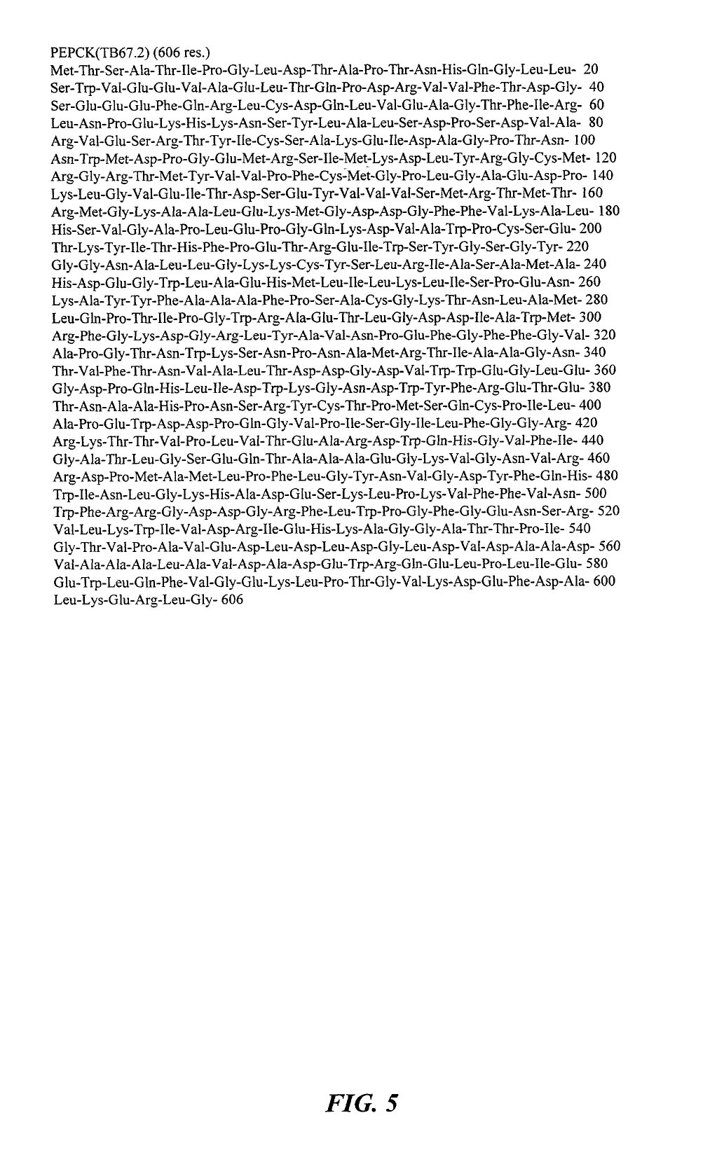

As noted above, the present invention is generally directed to compounds and methods for the diagnosis and therapy of M. tuberculosis infection. This invention is based, in part, on the discovery of two M. tuberculosis antigens (Mtb-81 and Mtb-67.2). Compounds provided herein include Mtb-81 polypeptides, which comprise at least an immunogenic portion of Mtb-81 or a variant thereof, and Mtb-67.2 polypeptides, which comprise at least an immunogenic portion of Mtb-67.2 or a variant thereof. Mtb-81 is an 81 kD M. tuberculosis antigen having the sequence recited in SEQ ID NO:2 and FIG. 2. Mtb-67.2 has the sequence recited in SEQ ID NO:5 and FIG. 5. Nucleic acid sequences encoding at least a portion of such polypeptides (or complements of such nucleic acid sequences) are also provided. Compounds provided herein also include binding agents such as antibodies (i.e., immune system proteins, or antigen-binding fragments thereof). Mtb-81 and Mt-67.2 polypeptides, polynucleotides and antibodies may be used within a variety of serodiagnostic methods for tuberculosis detection, and provide enhanced sensitivity in patients infected with HIV. Such compounds may also be used for immunotherapy of tuberculosis.

MTB-81 And MTB-67.2 Polynucleotides

Any polynucleotide that encodes an Mtb-81 or Mtb-67.2 polypeptide, as described herein, is encompassed by the present invention. Preferred polynucleotides comprise at least 10 consecutive nucleotides, preferably at least 15 consecutive nucleotides, and more preferably at least 30 consecutive nucleotides, that encode a portion of Mtb-81 or Mtb-67.2. Within certain embodiments, a polynucleotide may encode an immunogenic portion of Mtb-81 or Mtb-67.2. Polynucleotides comprising at least 15 consecutive nucleotides complementary to any such sequences are also encompassed by the present invention. Polynucleotides may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous M. tuberculosis sequence that encodes Mtb-81, Mtb-67.2 or a portion thereof) or may comprise a variant of such a sequence. Certain polynucleotide variants may contain one or more substitutions, additions, deletions and/or insertions such that the immunogenicity of the encoded polypeptide is not diminished, relative to native Mtb-81 or Mtb-67.2. The effect on the immunogenicity of the encoded polypeptide may generally be assessed as described herein. Variants preferably exhibit at least about 70% identity, more preferably at least about 80% identity and most preferably at least about 90% identity to a native polynucleotide sequence that encodes Mtb-81, Mtb-67.2 or a portion thereof. The percent identity may be readily determined by comparing sequences using computer algorithms well known to those of ordinary skill in the art, such as Megalign, using default parameters. Certain variants are substantially homologous to a native gene, or a portion or complement thereof. Such polynucleotide variants are capable of hybridizing under moderately stringent conditions to a naturally occurring DNA sequence. Suitable moderately stringent conditions include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

It will be appreciated by those of ordinary skill in the art that, as a result of the degeneracy of the genetic code, there are many nucleotide sequences that encode an Mtb-81 or Mtb-67.2 polypeptide as described herein. Some of these polynucleotides bear minimal homology to the nucleotide sequence of any native gene. Nonetheless, polynucleotides that vary due to differences in codon usage are specifically contemplated by the present invention.

Polynucleotides may be prepared using any of a variety of techniques. For example, a polynucleotide may be amplified via polymerase chain reaction (PCR) from cDNA or genomic DNA prepared from M. tuberculosis. For this approach, sequence-specific primers may be designed based on the sequences provided herein, and may be purchased or synthesized.

An amplified portion may be used to isolate a full length gene from a suitable library (e.g., an M. tuberculosis genomic or cDNA library) using well known techniques. Within such techniques, a library is screened using one or more polynucleotide probes or primers suitable for amplification. Preferably, a library is size-selected to include larger molecules. Random primed libraries may also be preferred for identifying 5′ and upstream regions of genes.

For hybridization techniques, a partial sequence may be labeled (e.g., by nick-translation or end-labeling with ³²P) using well known techniques. A bacterial or bacteriophage library is then screened by hybridizing filters containing denatured bacterial colonies (or lawns containing phage plaques) with the labeled probe (see Sambrook et al., Molecular Cloning: A Laboratory Manual, Cold Spring Harbor Laboratories, Cold Spring Harbor, N.Y., 1989). Hybridizing colonies or plaques are selected and expanded, and the DNA is isolated for further analysis. cDNA clones may be analyzed to determine the amount of additional sequence by, for example, PCR using a primer from the partial sequence and a primer from the vector. Restriction maps and partial sequences may be generated to identify one or more overlapping clones. The complete sequence may then be determined using standard techniques, which may involve generating a series of deletion clones. The resulting overlapping sequences are then assembled into a single contiguous sequence. A full length cDNA molecule can be generated by ligating suitable fragments, using well known techniques.

Alternatively, there are numerous amplification techniques for obtaining a full length coding sequence from a partial cDNA sequence. Within such techniques, amplification is generally performed via PCR. Any of a variety of commercially available kits may be used to perform the amplification step. Primers may be designed using, for example, software well known in the art. Primers are preferably 22-38 nucleotides in length, have a GC content of at least 50% and anneal to the target sequence at temperatures of about 56° C. to 72° C. The amplified region may be sequenced as described above, and overlapping sequences assembled into a contiguous sequence.

One such amplification technique is inverse PCR (see Triglia et al., Nucl. Acids Res. 16:8186, 1988), which uses restriction enzymes to generate a fragment in the known region of the gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WO 96/38591. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic. 1:111-19, 1991) and walking PCR (Parker et al., Nucl. Acids. Res. 19:3055-60, 1991). Other methods employing amplification may also be employed to obtain a full length cDNA sequence.

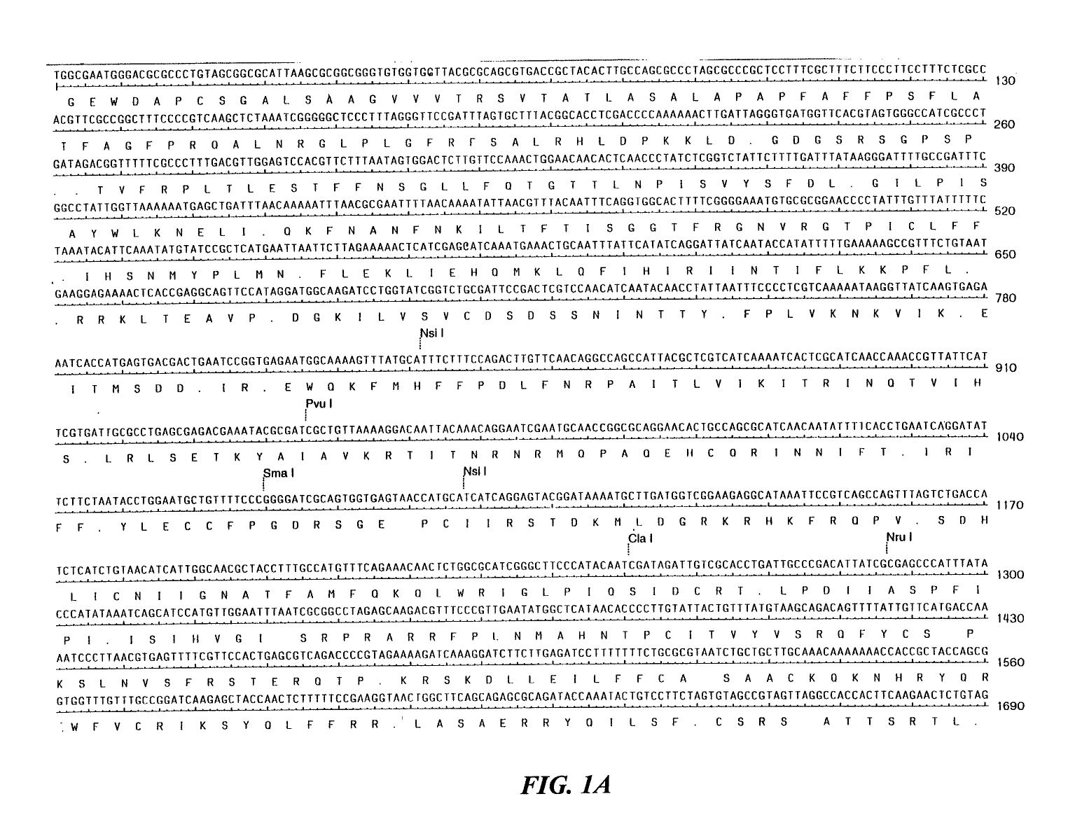

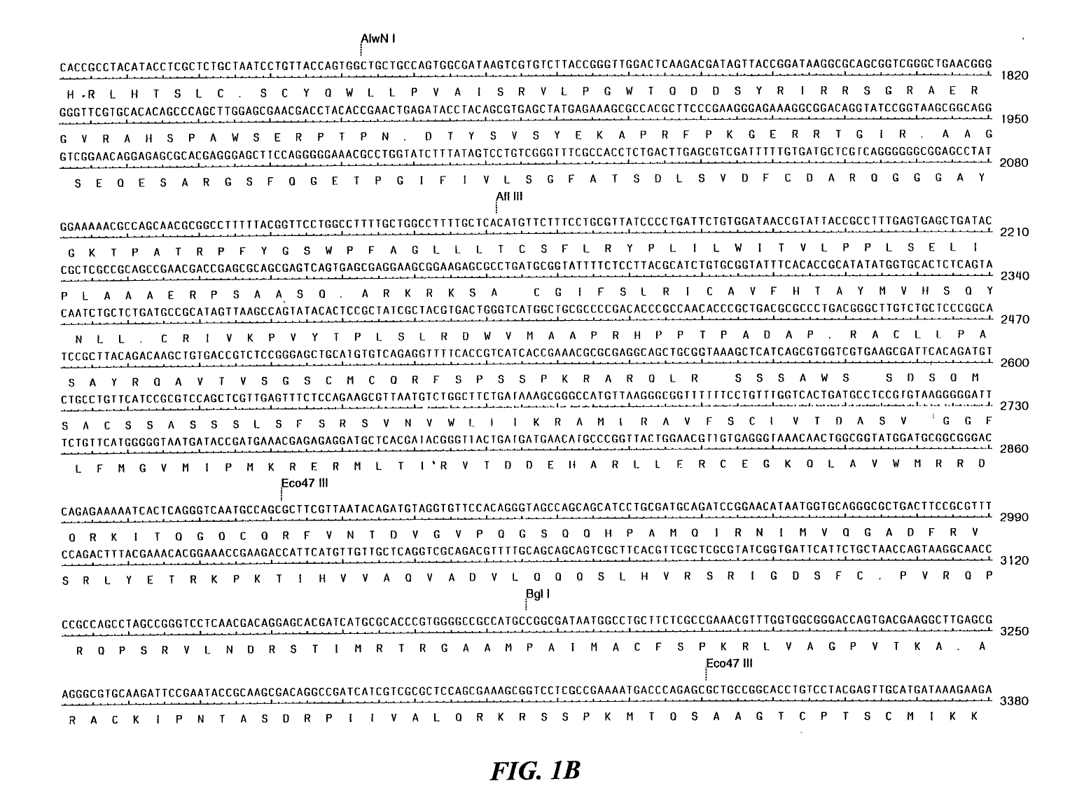

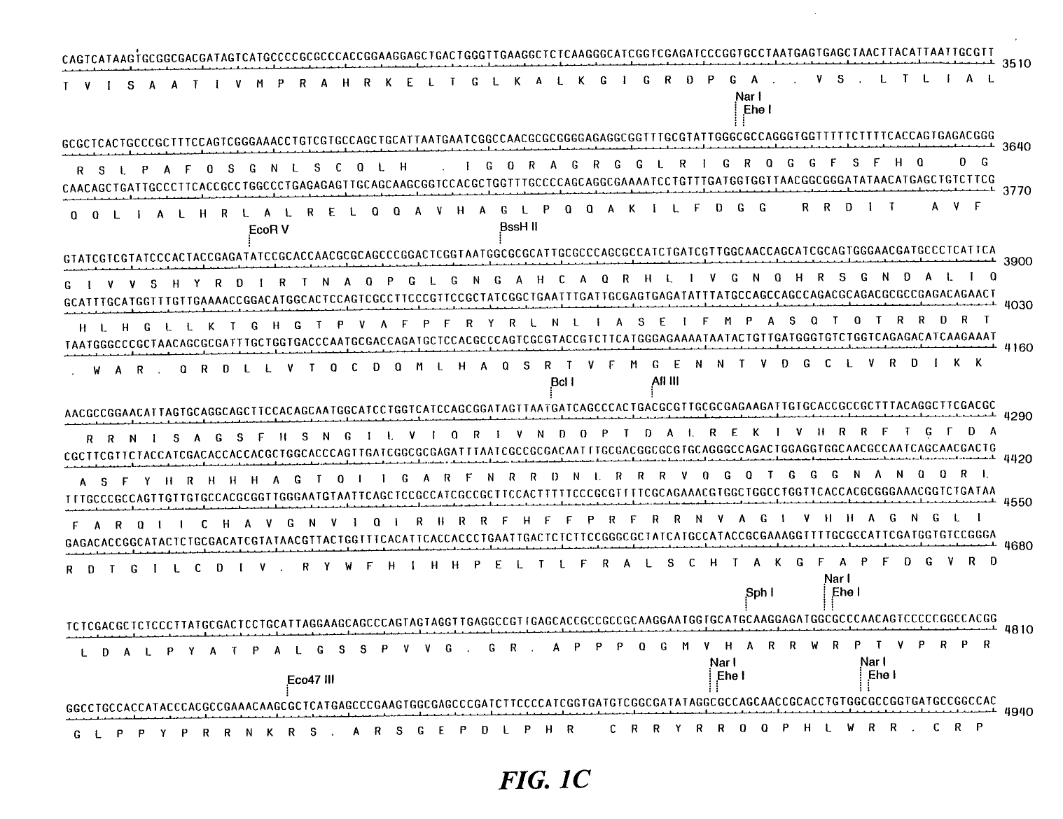

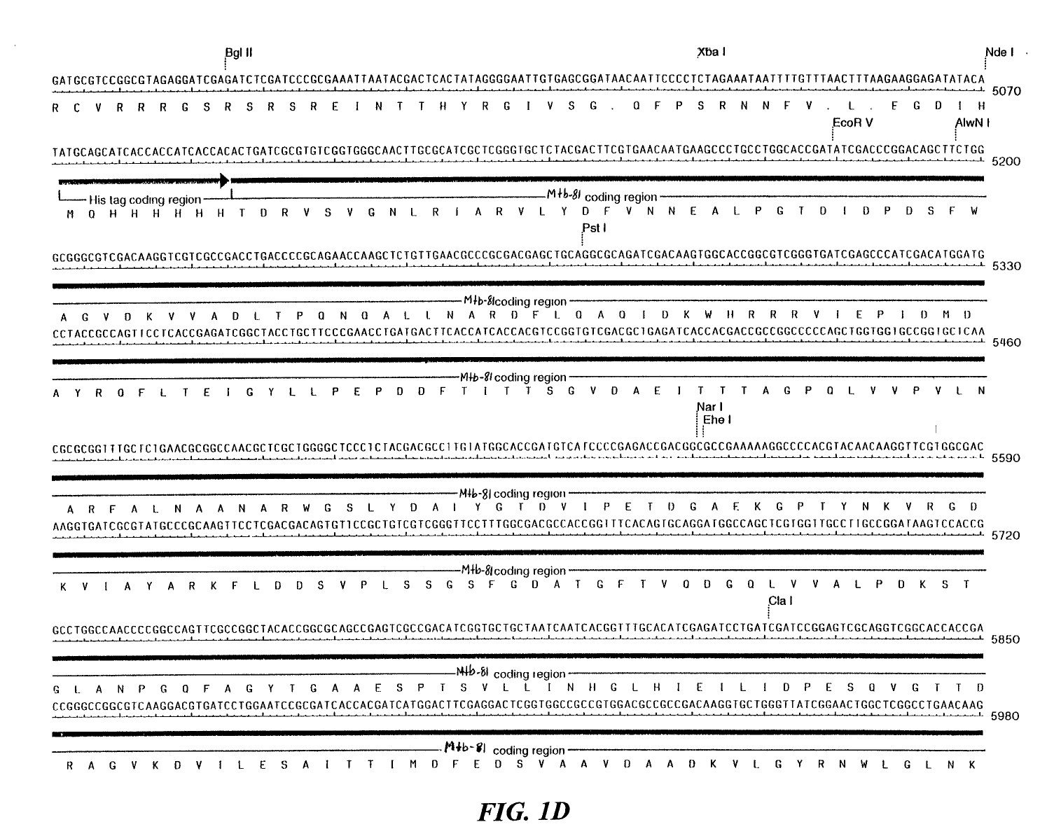

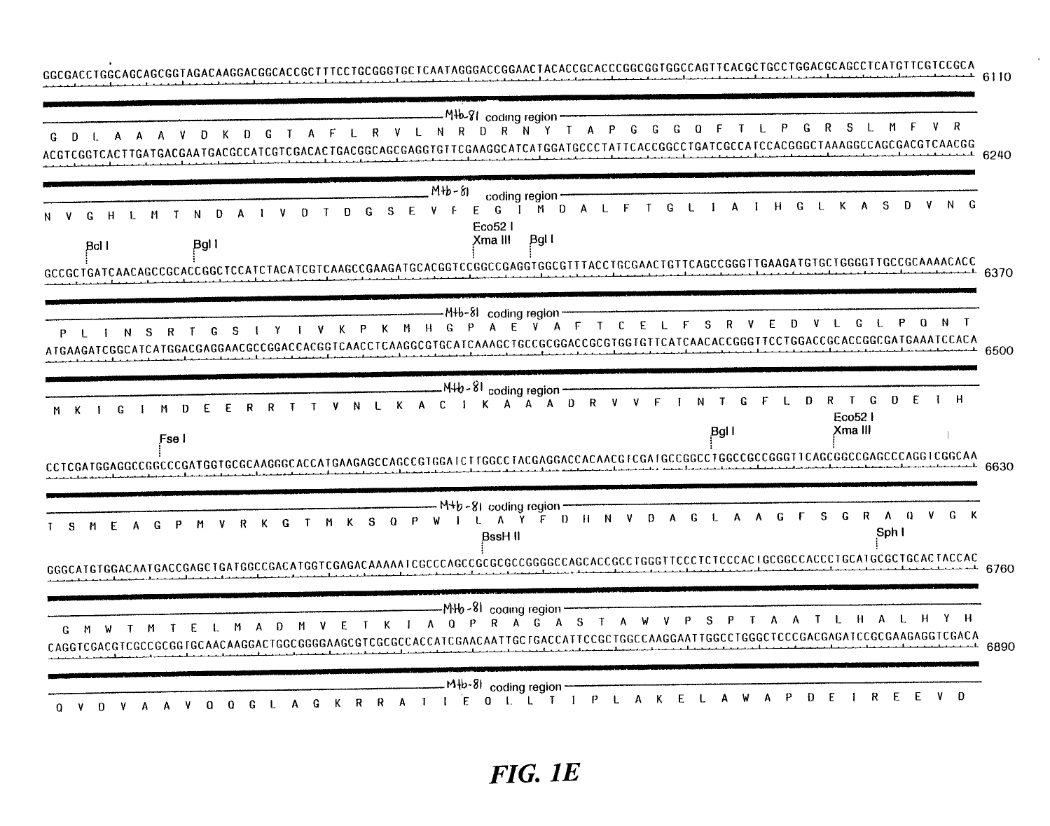

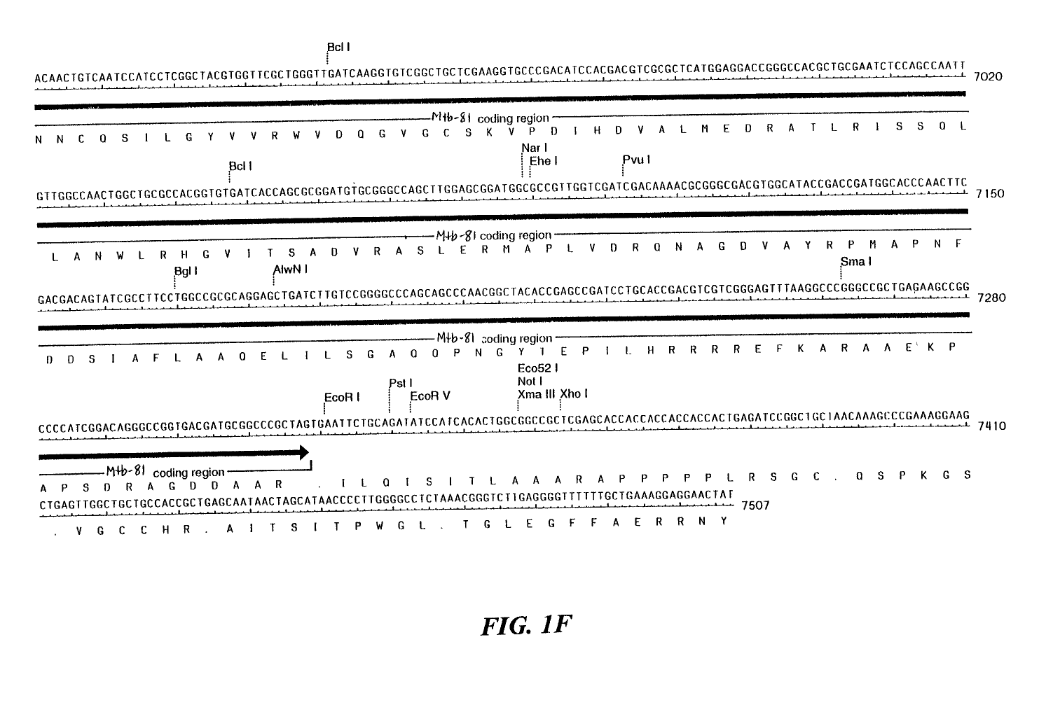

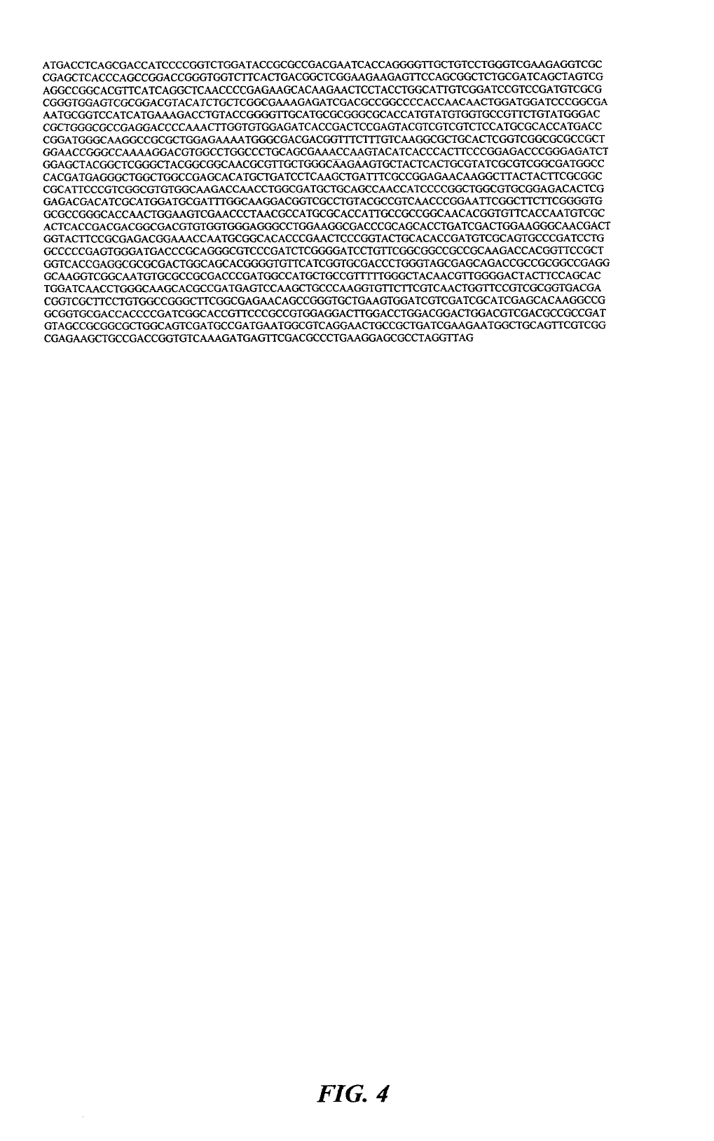

A genomic M. tuberculosis DNA sequence that includes the coding region Mtb-81 is presented in FIG. 1 (SEQ ID NO:3). In this figure, encoded amino acid residues are also indicated (SEQ ID NO:2), with the coding region for Mtb-81 (SEQ ID NO:1) indicated by the solid black bar. A DNA sequence (SEQ ID NO:4) encoding Mtb-67.2 is presented in FIG. 4, and the encoded amino acid residues are shown in FIG. 5 (SEQ ID NO:5). These coding regions, as well as portions thereof and sequences complementary to all or a portion thereof, are specifically encompassed by the present invention.

Polynucleotide variants may generally be prepared by any method known in the art, including chemical synthesis by, for example, solid phase phosphoramidite chemical synthesis. Modifications in a polynucleotide sequence may also be introduced using standard mutagenesis techniques, such as oligonucleotide-directed site-specific mutagenesis (see Adelman et al., DNA 2:183, 1983). Certain portions may be used to prepare an encoded polypeptide, as described herein. A portion of a coding sequence or a complementary sequence may also be designed as a probe or primer to detect gene expression. Probes may be labeled by a variety of reporter groups, such as radionuclides and enzymes, and are preferably at least 15 nucleotides in length, more preferably at least 30 nucleotides in length and still more preferably at least 50 nucleotides in length. Primers, as noted above, are preferably 22-38 nucleotides in length.

Any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Nucleotide sequences as described herein may be joined to a variety of other nucleotide sequences using established recombinant DNA techniques. For example, a polynucleotide may be cloned into any of a variety of cloning vectors, including plasmids, phagemids, lambda phage derivatives and cosmids. Vectors of particular interest include expression vectors, replication vectors, probe generation vectors and sequencing vectors. In general, a vector will contain an origin of replication functional in at least one organism, convenient restriction endonuclease sites and one or more selectable markers. Other elements will depend upon the desired use, and will be apparent to those of ordinary skill in the art.

Within certain embodiments, polynucleotides may be formulated so as to permit entry into a cell of a mammal, and expression therein. Such formulations are particularly useful for therapeutic purposes, as described below. Those of ordinary skill in the art will appreciate that there are many ways to achieve expression of a polynucleotide in a target cell, and any suitable method may be employed. For example, a polynucleotide may be incorporated into a viral vector such as, but not limited to, adenovirus, adeno-associated virus, retrovirus, or vaccinia or other pox virus (e.g., avian pox virus). Techniques for incorporating DNA into such vectors are well known to those of ordinary skill in the art. A retroviral vector may additionally transfer or incorporate a gene for a selectable marker (to aid in the identification or selection of transduced cells) and/or a targeting moiety, such as a gene that encodes a ligand for a receptor on a specific target cell, to render the vector target specific. Targeting may also be accomplished using an antibody, by methods known to those of ordinary skill in the art.

Other formulations for therapeutic purposes include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

MTB-81 and MTB-67.2 Polypeptides

Within the context of the present invention, Mtb-81 polypeptides comprise at least an immunogenic portion of Mtb-81 (FIGS. I A-IF; SEQ ID NO:2) or a variant thereof, as described herein. Mtb-67.2 polypeptides comprise at least an immunogenic portion of Mtb-67.2 (FIG. 5; SEQ ID NO:5) or a variant thereof. Polypeptides as described herein may be of any length. Additional sequences derived from the native protein and/or heterologous sequences may be present, and such sequences may (but need not) possess further immunogenic or antigenic properties.

An “immunogenic portion,” as used herein is a portion of an antigen that is recognized (i.e., specifically bound) by a B-cell and/or T-cell surface antigen receptor. Such immunogenic portions generally comprise at least 5 amino acid residues, preferably at least 9, more preferably at least 15, and still more preferably at least 50 amino acid residues of Mtb-81, Mtb-67.2 or a variant of either antigen. Immunogenic portions may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, antisera and/or T-cell lines or clones. As used herein, antisera and antibodies are “antigen-specific” if they specifically bind to an antigen (i.e., they react with the antigen in an ELISA or other immunoassay, and do not react detectably with unrelated proteins). Such antisera and antibodies may be prepared as described herein, and using well known techniques. An immunogenic portion of-Mtb-81 or Mtb-67.2 is a portion that reacts with such antisera and/or T-cells at a level that is not substantially less than the reactivity of the full length polypeptide (e.g., in an ELISA and/or T-cell reactivity assay). Immunogenic portions may react within such assays at a level that is similar to or greater than the reactivity of the full length polypeptide. Such screens may generally be performed using methods well known to those of ordinary skill in the art, such as those described in Harlow and Lane, Antibodies: A Laboratory Manual, Cold Spring Harbor Laboratory, 1988. For example, a polypeptide may be immobilized on a solid support and contacted with patient sera to allow binding of antibodies within the sera to the immobilized polypeptide. Unbound sera may then be removed and bound antibodies detected using a detection reagent, such as ¹²⁵I-labeled Protein A.

As noted above, a polypeptide may be a variant of Mtb-81 or Mtb-67.2. A polypeptide “variant,” as used herein, is a polypeptide that differs from native Mtb-81 or Mtb-67.2 in one or more substitutions, deletions, additions and/or insertions, such that the immunogenicity of the polypeptide is not substantially diminished. In other words, the ability of a variant to react with antigen-specific antisera or T cells may be enhanced or unchanged, relative to the native antigen, or may be diminished by less than 50%, and preferably less than 20%, relative to the native antigen. Such variants may generally be identified by modifying one of the above polypeptide sequences and evaluating the reactivity of the modified polypeptide with antigen-specific antibodies or antisera as described herein. Polypeptide variants preferably exhibit at least 70%, more preferably at least 90% and most preferably at least 95% identity to Mtb-81 or Mtb-67.2.

Preferably, a variant contains conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. Variants may also (or alternatively) be modified by, for example, the deletion or addition of amino acids that have minimal influence on the immunogenicity, secondary structure and hydropathic nature of the polypeptide.

As noted above, polypeptides may comprise a signal (or leader) sequence at the N-terminal end of the protein which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

Polypeptides may be prepared using any of a variety of well known techniques. Recombinant polypeptides encoded by DNA sequences as described above may be readily prepared from the DNA sequences using any of a variety of expression vectors known to those of ordinary skill in the art. Expression may be achieved in any appropriate host cell that has been transformed or transfected with an expression vector containing a DNA molecule that encodes a recombinant polypeptide. Suitable host cells include prokaryotes, yeast and higher eukaryotic cells. Preferably, the host cells employed are E. coli, yeast or a mammalian cell line such as COS or CHO. Supernatants from suitable host/vector systems which secrete recombinant protein or polypeptide into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant polypeptide.

Portions and other variants having fewer than about 100 amino acids, and generally fewer than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Applied BioSystems, Inc. (Foster City, Calif.), and may be operated according to the manufacturer's instructions.

Within certain specific embodiments, a polypeptide may be a fusion protein that comprises a polypeptide as described herein. For example, such fusion proteins may further comprise one or more known M. tuberculosis antigens, or variant(s) of such antigens. Representative known M. tuberculosis antigens include the 38 kD antigen described in Andersen and Hansen, Infect. Immun. 57:2481-2488, 1989 (GenBank Accession No. M30046) and ESAT-6 (Sorensen et al., Infect. Immun. 63:1710-1717, 1995). Fusion proteins may generally be prepared using standard techniques. For example, a fusion protein may be prepared recombinantly. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and the second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

Fusion proteins are also provided that comprise a polypeptide of the present invention together with an unrelated immunogenic protein. Preferably the immunogenic protein is capable of eliciting a recall response. Examples of such proteins include tetanus, tuberculosis and hepatitis proteins (see, for example, Stoute et al. New Engl. J. Med., 336:86-91, 1997).

In general, polypeptides (including fusion proteins) and polynucleotides as described herein are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

Binding Agents

The present invention further provides agents, such as antibodies and antigen-binding fragments thereof, that specifically bind to Mtb-81 or Mtb-67.2. As used herein, an antibody, or antigen-binding fragment thereof, is said to “specifically bind” to Mtb-81 or Mtb-67.2 if it reacts at a detectable level (within, for example, an ELISA) with Mtb-81 or Mtb-67.2, and does not react detectably with unrelated proteins under similar conditions. As used herein, “binding” refers to a noncovalent association between two separate molecules (each of which may be in solution or present on the surface of a cell or solid support) such that a “complex” is formed. The ability to bind may be evaluated by, for example, determining a binding constant for the formation of the complex. The binding constant is the value obtained when the concentration of the complex is divided by the product of the component concentrations. In general, two compounds are said to “bind,” in the context of the present invention, when the binding constant for complex formation exceeds about 10³ L/mol. The binding constant maybe determined using methods well known in the art.

Binding agents are further capable of differentiating between patients with and without M. tuberculosis infection, using the representative assays provided herein. In other words, antibodies or other binding agents that bind to Mtb-81 or Mtb-67.2 will generate a signal indicating the presence of M. tuberculosis infection in at least about 20% of patients with such infection, and will generate a negative signal indicating the absence of such infection in at least about 90% of uninfected individuals. In general, a signal is considered positive if it is greater than the mean signal obtained from an uninfected sample plus three standard deviations. To determine whether a binding agent satisfies this requirement, biological samples (e.g., blood, sera, plasma, saliva, cerebrospinal fluid or urine) from patients with and without M. tuberculosis infection (as determined using a standard diagnostic test) may be assayed as described herein for the presence of polypeptides that bind to the binding agent. It will be apparent that a statistically significant number of samples with and without the infection should be assayed. Each binding agent should satisfy the above criteria; however, those of ordinary skill in the art will recognize that binding agents may be used in combination to improve sensitivity.

Any agent that satisfies the above requirements may be a binding agent. For example, a binding agent may be a ribosome with or without a peptide component, an RNA molecule or a polypeptide. In a preferred embodiment, a binding agent is an antibody or an antigen-binding fragment thereof. Such antibodies may be polyclonal or monoclonal. In addition, the antibodies may be single chain, chimeric, CDR-grafted or humanized.

Binding agents may be further linked to a reporter group, to facilitate diagnostic assays. Suitable reporter groups will be apparent to those of ordinary skill in the art, and include enzymes (such as horseradish peroxidase), substrates, cofactors, inhibitors, dyes, colloids (e.g., colloidal gold), radionuclides, luminescent groups, fluorescent groups and biotin. The conjugation of antibody to reporter group may be achieved using standard methods known to those of ordinary skill in the art.

Antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies. To generate antibodies, a polypeptide immunogen may be the full length Mtb-81 or Mtb-67.2, or may be an immunogenic portion of either antigen. If an immunogenic portion is employed, the resulting antibody should indicate the presence of M. tuberculosis infection in substantially all (i.e., at least 80%, and preferably at least 90%) of the patients for which M. tuberculosis infection would be indicated using an antibody raised against the full length antigen. The antibody should also indicate the absence of M. tuberculosis infection in substantially all of those samples that would be negative when tested with an antibody raised against the full length antigen. The representative assays provided herein, such as the two-antibody sandwich assay, may generally be employed for evaluating the ability of an antibody to detect tuberculosis.

In one technique, an immunogen comprising the polypeptide is initially injected into any of a wide variety of mammals (e.g., mice, rats, rabbits, sheep or goats). In this step, the polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for the antigenic polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

Within certain embodiments, the use of antigen-binding fragments of antibodies may be preferred. Such fragments include Fab fragments, which may be prepared using standard techniques. Briefly, immunoglobulins may be purified from rabbit serum by affinity chromatography on Protein A bead columns (Harlow and Lane, Antibodies: A Laboratory Manual, Cold Spring Harbor Laboratory, 1988) and digested by papain to yield Fab and Fc fragments. The Fab and Fc fragments may be separated by affinity chromatography on protein A bead columns.

Monoclonal antibodies of the present invention may be coupled to one or more therapeutic agents for use in the therapeutic methods provided herein. Suitable agents in this regard include radionuclides, differentiation inducers, drugs, toxins, and derivatives thereof. Preferred radionuclides include ⁹⁰Y, ¹²³I, ¹²⁵I, ¹³¹I, ¹⁸⁶Re, ¹⁸⁸Re, ²¹¹At, and ²¹²Bi. Preferred drugs include methotrexate, and pyrimidine and purine analogs. Preferred differentiation inducers include phorbol esters and butyric acid. Preferred toxins include ricin, abrin, diptheria toxin, cholera toxin, gelonin, Pseudomonas exotoxin, Shigella toxin, and pokeweed antiviral protein.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

Where a therapeutic agent is more potent when free from the antibody portion of the immunoconjugates of the present invention, it may be desirable to use a linker group which is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers which provide multiple sites for attachment can be used. Alternatively, a carrier can be used.

A carrier may bear the agents in a variety of ways, including covalent bonding either directly or via a linker group. Suitable carriers include proteins such as albumins (e.g., U.S. Pat. No. 4,507,234, to Kato et al.), peptides and polysaccharides such as aminodextran (e.g., U.S. Pat. No. 4,699,784, to Shih et al.). A carrier may also bear an agent by noncovalent bonding or by encapsulation, such as within a liposome vesicle (e.g., U.S. Pat. Nos. 4,429,008 and 4,873,088). Carriers specific for radionuclide agents include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis.

A variety of routes of administration for the antibodies and immunoconjugates may be used. Typically, administration will be intravenous, intramuscular or subcutaneous. It will be evident that the precise dose of the antibody/immunoconjugate will vary depending upon the antibody used and the rate of clearance of the antibody.

### Methods for Detecting Tuberculosis

In general, M. tuberculosis infection may be detected in a patient based on the presence of one or more of the following in a biological sample obtained from a patient: (a) antibodies that specifically bind to Mtb-81 or Mtb-67.2; (b) T-cells that specifically react with Mtb-81 or Mtb-67.2; (c) Mtb-81 or Mtb-67.2 antigen or (d) mRNA encoding Mtb-81 or Mtb-67.2 antigen. In other words, Mtb-81 and/or Mtb-67.2 may be used as a marker to indicate the presence or absence of M. tuberculosis infection in a patient. Mtb-81 or Mtb-67.2 polypeptides, as well as polynucleotides encoding such polypeptides and antigen-presenting cells that express such polypeptides, may be used to detect the presence of specific antibodies or T-cells. The binding agents provided herein generally permit detection of the level of Mtb-81 or Mtb-67.2 antigen in the biological sample. Polynucleotide primers and probes may be used to detect the level of mRNA encoding Mtb-81 or Mtb-67.2.

Diagnostic methods provided herein have advantages over existing methods in sensitivity. In particular, methods provided herein may be used to detect M. tuberculosis infection in AIDS patients. M. tuberculosis and HIV co-infection is common in such patients, but the tuberculosis has been difficult to detect using previous diagnostic methods. Further, Mtb-81 appears to be an early stage marker for M. tuberculosis infection, permitting early detection of the disease.

A biological sample may be any sample obtained from one or more human or non-human animals that would be expected to contain the target substance in infected individuals. For example, to detect M. tuberculosis infection based on the presence of Mtb-81- or Mtb-67.2-specific antibodies, any antibody-containing sample may be used. Such samples include whole blood, sputum, serum, plasma, saliva, cerebrospinal fluid and urine. Preferred biological samples include blood, serum and plasma obtained from a patient or blood supply.

Within the methods provided herein, Mtb-81 and/or Mtb-67.2 may, but need not, be used in combination with one or more known M. tuberculosis antigens. In such embodiments, the antigens used are preferably complementary (i.e., one antigen will tend to detect infection in samples where the infection would not be detected by the other antigen). Complementary antigens may generally be identified by using each polypeptide individually to evaluate serum samples obtained from a series of patients known to be infected with M. tuberculosis. After determining which samples test positive (as described below) with each polypeptide, combinations of two or more polypeptides may be formulated that are capable of detecting infection in most, or all, of the samples tested. Such polypeptides are complementary.

There are a variety of assay formats known to those of ordinary skill in the art for using one or more polypeptides to detect antibodies in a sample. See, e.g., Harlow and Lane, Antibodies: A Laboratory Manual, Cold Spring Harbor Laboratory, 1988, which is incorporated herein by reference. In a preferred embodiment, the assay involves the use of polypeptide immobilized on a solid support to bind to and remove the antibody (in the form of an immunocomplex with polypeptide) from the sample. The immunocomplex may then be detected using a detection reagent that contains a reporter group. Suitable detection reagents include antibodies that bind to the immunocomplex and free polypeptide labeled with a reporter group (e.g., in a semi-competitive assay). Alternatively, a competitive assay may be utilized, in which an antibody that binds to the polypeptide is labeled with a reporter group and allowed to bind to the immobilized antigen after incubation of the antigen with the sample. The extent to which components of the sample inhibit the binding of the labeled antibody to the polypeptide is indicative of the reactivity of the sample with the immobilized polypeptide.

The solid support may be any solid material known to those of ordinary skill in the art to which the antigen may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681.

The polypeptides may be bound to the solid support using a variety of techniques known to those of ordinary skill in the art, which are amply described in the patent and scientific literature. In the context of the present invention, the term “bound” refers to both noncovalent association, such as adsorption, and covalent attachment (which may be a direct linkage between the antigen and functional groups on the support or may be a linkage by way of a cross-linking agent). Binding by adsorption to a well in a microtiter plate or to a membrane is preferred. In such cases, adsorption may be achieved by contacting the polypeptide, in a suitable buffer, with the solid support for a suitable amount of time. The contact time varies with temperature, but is typically between about 1 hour and 1 day. In general, contacting a well of a plastic microtiter plate (such as polystyrene or polyvinylchloride) with an amount of polypeptide ranging from about 10 ng to about 1 μg, and preferably about 100 ng, is sufficient to bind an adequate amount of antigen.

Covalent attachment of polypeptide to a solid support may generally be achieved by first reacting the support with a bifunctional reagent that will react with both the support and a functional group, such as a hydroxyl or amino group, on the polypeptide. For example, the polypeptide may be bound to supports having an appropriate polymer coating using benzoquinone or by condensation of an aldehyde group on the support with an amine and an active hydrogen on the polypeptide (see, e.g., Pierce Immunotechnology Catalog and Handbook, 1991, at A12-A13).

In certain embodiments, the assay is an enzyme linked immunosorbent assay (ELISA). This assay may be performed by first contacting a polypeptide antigen that has been immobilized on a solid support, commonly the well of a microtiter plate, with the sample, such that antibodies to the polypeptide within the sample are allowed to bind to the immobilized polypeptide. Unbound sample is then removed from the immobilized polypeptide and a detection reagent capable of binding to the immobilized immunocomplex is added. The amount of detection reagent that remains bound to the solid support is then determined using a method appropriate for the specific detection reagent.

More specifically, once the polypeptide is immobilized on the support as described above, the remaining protein binding sites on the support are typically blocked. Any suitable blocking agent known to those of ordinary skill in the art, such as bovine serum albumin or Tween 20™ (Sigma Chemical Co., St. Louis, Mo.) may be employed. The immobilized polypeptide is then incubated with the sample, and antibody is allowed to bind to the antigen. The sample may be diluted with a suitable diluent, such as phosphate-buffered saline (PBS) prior to incubation. In general, an appropriate contact time (i.e., incubation time) is that period of time that is sufficient to detect the presence of antibody within a M. tuberculosis-infected sample. Preferably, the contact time is sufficient to achieve a level of binding that is at least 95% of that achieved at equilibrium between bound and unbound antibody. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. Detection reagent may then be added to the solid support. An appropriate detection reagent is any compound that binds to the immobilized antibody-polypeptide complex and that can be detected by any of a variety of means known to those in the art. Preferably, the detection reagent contains a binding agent (such as, for example, Protein A, Protein G, immunoglobulin, lectin or free antigen) conjugated to a reporter group. Preferred reporter groups include enzymes (such as horseradish peroxidase), substrates, cofactors, inhibitors, colloids, dyes, radionuclides, luminescent groups, fluorescent groups and biotin. The conjugation of binding agent to reporter group may be achieved using standard methods known to those of ordinary skill in the art. Common binding agents may also be purchased conjugated to a variety of reporter groups from many commercial sources (e.g., Zymed Laboratories, San Francisco, Calif., and Pierce, Rockford, Ill.).

The detection reagent is then incubated with the immobilized antibody-polypeptide complex for an amount of time sufficient to detect the bound antibody. An appropriate amount of time may generally be determined from the manufacturer's instructions or by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of anti-M. tuberculosis antibodies in the sample, the signal detected from the reporter group that remains bound to the solid support is generally compared to a signal that corresponds to a cut-off value. In one preferred embodiment, the cut-off value is the average mean signal plus three standard deviations obtained when the immobilized antigen is incubated with samples from an uninfected patient. In general, a sample generating a signal that is above the cut-off value is considered positive for tuberculosis. In an alternate preferred embodiment, the cut-off value is determined using a Receiver Operator Curve, according to the method of Sackett et al., Clinical Epidemiology: A Basic Science for Clinical Medicine, Little Brown and Co., 1985, pp. 106-107. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for tuberculosis.

In a related embodiment, the assay is performed in a rapid flow-through or strip test format, wherein the antigen is immobilized on a membrane, such as nitrocellulose. In the flow-through test, antibodies within the sample bind to the immobilized polypeptide as the sample passes through the membrane. A detection reagent (e.g., protein A-colloidal gold) then binds to the antibody-polypeptide complex as the solution containing the detection reagent flows through the membrane. The detection of bound detection reagent may then be performed as described above. In the strip test format, one end of the membrane to which polypeptide is bound is immersed in a solution containing the sample. The sample migrates along the membrane through a region containing detection reagent and to the area of immobilized polypeptide. Concentration of detection reagent at the polypeptide indicates the presence of anti-M. tuberculosis antibodies in the sample. Typically, the concentration of detection reagent at that site generates a pattern, such as a line, that can be read visually. The absence of such a pattern indicates a negative result. In general, the amount of polypeptide immobilized on the membrane is selected to generate a visually discernible pattern when the biological sample contains a level of antibodies that would be sufficient to generate a positive signal in an ELISA, as discussed above. Preferably, the amount of polypeptide immobilized on the membrane ranges from about 25 ng to about 1 μg, and more preferably from about 50 ng to about 500 ng. Such tests can typically be performed with a very small amount (e.g., one drop) of patient serum or blood.

Of course, numerous other assay protocols exist that are suitable for use with the polypeptides of the present invention. The above descriptions are intended to be exemplary only. For example, it will be apparent to those of ordinary skill in the art that the above protocols may be readily modified to use antibodies, or antigen-binding fragments thereof, to detect Mtb-81 and/or Mtb-67.2 in a biological sample.

M. tuberculosis infection may also, or alternatively, be detected based on the presence of T cells that specifically react with Mtb-81 in a biological sample. Within certain methods, a biological sample comprising CD4⁺ and/or CD8⁺ T cells isolated from a patient is incubated with a Mtb-81 or Mtb-67.2 polypeptide, a polynucleotide encoding such a polypeptide and/or an APC that expresses such al polypeptide, and the presence or absence of specific activation of the T cells is detected. Suitable biological samples include, but are not limited to, isolated T cells. For example, T cells may be isolated from a patient by routine techniques (such as by Ficoll/Hypaque density gradient centrifugation of peripheral blood lymphocytes). T cells may be incubated in vitro for 2-9 days (typically 4 days) at 37° C. with Mtb-81 or Mtb-67.2 polypeptide (e.g., 5-25 μg/ml). It may be desirable to incubate another aliquot of a T cell sample in the absence of Mtb-81 or Mtb-67.2 polypeptide to serve as a control. For CD4⁺ T cells, activation is preferably detected by evaluating proliferation of the T cells. For CD8⁺ T cells, activation is preferably detected by evaluating cytolytic activity. A level of proliferation that is at least two fold greater and/or a level of cytolytic activity that is at least 20% greater than in disease-free patients indicates the presence of M. tuberculosis infection.

As noted above, M. tuberculosis infection may also, or alternatively, be detected based on the level of mRNA encoding Mtb-81 or Mtb-67.2 in a biological sample. For example, at least two oligonucleotide primers may be employed in a polymerase chain reaction (PCR) based assay to amplify a portion Mtb-81 or Mtb-67.2 cDNA derived from a biological sample, wherein at least one of the oligonucleotide primers is specific for (i.e., hybridizes to) a polynucleotide encoding Mtb-81 or Mtb-67.2. The amplified cDNA is then separated and detected using techniques well known in the art, such as gel electrophoresis. Similarly, oligonucleotide probes that specifically hybridize to a polynucleotide encoding Mtb-81 or Mtb-67.2 may be used in a hybridization assay to detect the presence of polynucleotide encoding the antigen in a biological sample.

To permit hybridization under assay conditions, oligonucleotide primers and probes should comprise an oligonucleotide sequence that has at least about 60%. preferably at least about 75% and more preferably at least about 90%, identity to a portion of a polynucleotide encoding Mtb-81 or Mtb-67.2 that is at least 10 nucleotides, and preferably at least 20 nucleotides, in length. Oligonucleotide primers and/or probes which may be usefully employed in the diagnostic methods described herein are preferably at least 10-40 nucleotides in length. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, NY, 1989).

One preferred assay employs RT-PCR, in which PCR is applied in conjunction with reverse transcription. Typically, RNA is extracted from a sample tissue and is reverse transcribed to produce cDNA molecules. PCR amplification using at least one specific primer generates a cDNA molecule, which may be separated and visualized using, for example, gel electrophoresis. Amplification may be performed on samples obtained from biological samples taken from a test patient and an individual who is not infected with M. tuberculosis. The amplification reaction may be performed on several dilutions of cDNA spanning two orders of magnitude. A two-fold or greater increase in expression in several dilutions of the test patient sample as compared to the same dilutions of an uninfected sample is typically considered positive.

As noted above, to improve sensitivity, multiple M. tuberculosis markers may be assayed within a given sample. It will be apparent that multiple antigens may be combined within a single assay, or multiple primers or probes may be used concurrently. The selection of antigen markers may be based on routine experiments to determine combinations that results in optimal sensitivity.

The diagnostic methods provided above may be used to monitor tuberculosis therapy in a patient. Briefly, such monitoring may be achieved by performing an assay as described above using a biological sample obtained at a first time (prior to at least a portion of a therapy), and comparing the result obtained with the result of a similar assay performed using a second biological sample (obtained following at least a portion of the therapy). A therapy that results in a decrease in signal is generally considered to be effective in decreasing the level of M. tuberculosis infection.

### Diagnositic Kits

The present invention further provides kits for use within any of the above diagnostic methods. Such kits typically comprise two or more components suitable for performing a diagnostic assay. Components may be compounds, reagents, containers and/or equipment. For example, one container within a kit may contain a Mtb-81 or Mtb-67.2 polypeptide. Such polypeptides may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of immunocomplex formation.

Alternatively, a kit may be designed to detect the level of mRNA encoding Mtb-81 or Mtb-67.2 in a biological sample. Such kits generally comprise at least one oligonucleotide probe or primer, as described above, that hybridizes to a polynucleotide encoding Mtb-81 or Mtb-67.2. Such an oligonucleotide may be used, for example, within a PCR or hybridization assay. Additional components that may be present within such kits include a second oligonucleotide and/or a diagnostic reagent or container to facilitate the detection of a polynucleotide encoding Mtb-81 or Mtb-67.2.

Still further kits may detect the presence of antigen in a sample. Such kits may comprise one or more monoclonal or polyclonal antibodies that specifically bind to Mtb-81 or Mtb-67.2.

### T Cells

The present invention further provides T cells specific for Mtb-81 or Mtb-67.2. Such cells may generally be prepared in vitro or ex vivo, using standard procedures. For example, T cells may be present within (or isolated from) bone marrow, peripheral blood or a fraction of bone marrow or peripheral blood of a mammal, such as a patient, using a commercially available cell separation system, such as the CEPRATE™ system, available from CellPro Inc. Bothell Wash. (see also U.S. Pat. No. 5,240,856; U.S. Pat. No. 5,215,926; WO 89/06280; WO 91/16116 and WO 92/07243). Alternatively, T cells may be derived from related or unrelated humans, non-human animals, cell lines or cultures.

T cells may be stimulated with a Mtb-81 or Mtb-67.2 polypeptide, a polynucleotide encoding such a polypeptide and/or an antigen presenting cell (APC) that expresses such a polypeptide. Stimulation is performed under conditions and for a time sufficient to permit the generation of T cells that are specific for the polypeptide. Preferably, a Mtb-81 or Mtb-67.2 polypeptide or polynucleotide is present within a delivery vehicle, such as a microsphere, to facilitate the generation of specific T cells.

T cells are considered to be specific for Mtb-81 (or Mtb-67.2) if the T cells kill target cells coated with Mtb-81 or expressing a gene encoding Mtb-81 (or Mtb-67.2). T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070, 1994. Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with Mtb-81 or Mtb-67.2 (200 ng/ml-100 μg/ml, preferably 100 ng/ml-25 μg/ml) for 3-7 days should result in at least a two fold increase in proliferation of the T cells and/or contact as described above for 2-3 hours should result in activation of the T cells, as measured using standard cytokine assays in which a two fold increase in the level of cytokine release (e.g., TNF or IFN-Y) is indicative of T cell activation (see Coligan et al., Current Protocols in Immunology, vol. 1, Wiley Interscience (Greene 1998)). T cells that have been activated in response to a Mtb-81 or Mtb-67.2 polypeptide, polynucleotide or polypeptide-expressing APC may be CD4⁺ and/or CD8⁺. Mtb-81- or Mtb-67.2-specific T cells may be expanded using standard techniques. Within preferred embodiments, the T cells are derived from a patient or a related or unrelated donor and are administered to the patient following stimulation and expansion.

For therapeutic purposes, CD4⁺ or CD8⁺ T cells that proliferate in response to Mtb-81 or Mtb-67.2 can be expanded in number either in vitro or in vivo. Proliferation of such T cells in vitro may be accomplished in a variety of ways. For example, the T cells can be re-exposed to Mtb-81 or Mtb-67.2, with or without the addition of T cell growth factors, such as interleukin-2, and/or stimulator cells that synthesize a Mtb-81 or Mtb-67.2 polypeptide. Alternatively, one or more T cells that proliferate in the presence of Mtb-81 or Mtb-67.2 can be expanded in number by cloning. Methods for cloning cells are well known in the art, and include limiting dilution.

### Pharmaceutical Compositions and Vaccines

Within certain aspects, polypeptides, polynucleotides, binding agents and/or cells may be incorporated into pharmaceutical compositions or vaccines. Pharmaceutical compositions comprise one or more such compounds and a physiologically acceptable carrier. Vaccines may comprise one or more such compounds and a non-specific immune response enhancer. A non-specific immune response enhancer may be any substance that enhances an immune response to an exogenous antigen. Examples of non-specific immune response enhancers include adjuvants, biodegradable microspheres (e.g., polylactic galactide) and liposomes (into which the compound is incorporated). Pharmaceutical compositions and vaccines within the scope of the present invention may also contain other compounds, which may be biologically active or inactive. For example, one or more immunogenic portions of other M. tuberculosis antigens may be present, either incorporated into a fusion polypeptide or as a separate compound within the composition or vaccine.

A pharmaceutical composition or vaccine may contain DNA encoding one or more of the polypeptides as described above, such that the polypeptide is generated in situ. DNA may be present within any of a variety of delivery systems known to those of ordinary skill in the art, including nucleic acid expression systems, bacteria and viral expression systems. Numerous gene delivery techniques are well known in the art, such as those described by Rolland, Crit. Rev. Therap. Drug Carrier Systems 15:143-198, 1998, and references cited therein. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface. In a preferred embodiment, the DNA may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502, 1993; Guzman et al., Circulation 88:2838-2848, 1993; and Guzman et al., Cir. Res. 73:1202-1207, 1993. Techniques for incorporating DNA into such expression systems are well known to those of ordinary skill in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

While any suitable carrier known to those of ordinary skill in the art may be employed in the pharmaceutical compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this invention. Suitable biodegradable microspheres are disclosed, for example, in U.S. Pat. Nos. 4,897,268 and 5,075,109.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions of the present invention may be formulated as a lyophilizate. Compounds may also be encapsulated within liposomes using well known technology.

Any of a variety of non-specific immune response enhancers may be employed in the vaccines of this invention. For example, an adjuvant may be included. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.), Merck Adjuvant 65 (Merck and Company, Inc., Rahway, N.J.), alum, biodegradable microspheres, monophosphoryl lipid A and quil A. Cytokines, such as GM-CSF or interleukin-2, -7, or -12, may also be used as adjuvants.

The compositions described herein may be administered as part of a sustained release formulation (i.e., a formulation such as a capsule or sponge that effects a slow release of compound following administration). Such formulations may generally be prepared using well known technology and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane. Carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

Any of a variety of delivery vehicles may be employed within pharmaceutical compositions and vaccines to facilitate production of an immune response. Delivery vehicles include antigen presenting cells, such as dendritic cells and macrophages. Such cells may be transfected with a polynucleotide encoding Mtb-81 or Mtb-67.2 (or portion or other variant thereof) such that the Mtb-81 or Mtb-67.2 polypeptide is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells may generally be performed using any methods known in the art, such as those described in WO 97/24447, or the gene gun approach described by Mahvi et al., Immunology and cell Biology 75:456-460, 1997.

### Tuberculosis Therapy

In further aspects of the present invention, the compositions described herein may be used for immunotherapy of tuberculosis. Within such methods, pharmaceutical compositions and vaccines are typically administered to a patient. As used herein, a “patient” refers to any warm-blooded animal, preferably a human. A patient may or may not be infected with M. tuberculosis. Accordingly, the above pharmaceutical compositions and vaccines may be used to prevent the development of tuberculosis or to treat a patient afflicted with tuberculosis. Pharmaceutical compositions and vaccines may be administered prior to, concurrent with or following treatment with other therapeutic agents.

Within certain embodiments, immunotherapy may be active immunotherapy, in which treatment relies on the in vivo stimulation of the endogenous host immune system to react against M. tuberculosis with the administration of immune response-modifying agents (such as tumor vaccines, bacterial adjuvants and/or cytokines).

Within other embodiments, immunotherapy may be passive immunotherapy, in which treatment involves the delivery of agents with established immune reactivity (such as effector cells or antibodies) that do not necessarily depend on an intact host immune system. Examples of effector cells include T lymphocytes (such as CD8⁺ cytotoxic T lymphocytes and CD4⁺ T-helper tumor-infiltrating lymphocytes), killer cells (such as Natural Killer cells and lymphokine-activated killer cells), B cells and antigen-presenting cells (such as dendritic cells and macrophages) expressing a polypeptide provided herein. T cell receptors and antibody receptors specific for the polypeptides recited herein may be cloned, expressed and transferred into other vectors or effector cells for adoptive immunotherapy. The polypeptides provided herein may also be used to generate antibodies or anti-idiotypic antibodies (as described above and in U.S. Pat. No. 4,918,164) for passive immunotherapy.

Effector cells may generally be obtained in sufficient quantities for adoptive immunotherapy by growth in vitro, as described herein. Culture conditions for expanding single antigen-specific effector cells to several billion in number with retention of antigen recognition in vivo are well known in the art. Such in vitro culture conditions typically use intermittent stimulation with antigen, often in the presence of cytokines (such as IL-2) and non-dividing feeder cells. As noted above, immunoreactive polypeptides as provided herein may be used to rapidly expand antigen-specific T cell cultures in order to generate a sufficient number of cells for immunotherapy. In particular, antigen-presenting cells, such as dendritic, macrophage or B cells, may be pulsed with immunoreactive polypeptides or transfected with one or more polynucleotides using standard techniques well known in the art. For example, antigen-presenting cells can be transfected with a polynucleotide having a promoter appropriate for increasing expression in a recombinant virus or other expression system. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL-2 (see, for example, Cheever et al., Immunological Reviews 157:177, 1997).

The polypeptides provided herein may also be used to generate and/or isolate Mtb-81- or Mtb-67.2-reactive T cells, which can then be administered to a patient. In one such technique, antigen-specific T cell lines may be generated by in vivo immunization with short peptides corresponding to immunogenic portions of the disclosed polypeptides. The resulting antigen-specific CD8⁺ CTL clones may be isolated from the patient, expanded using standard tissue culture techniques and returned to the patient.

Polypeptides may also be used for ex vivo treatment of tuberculosis. For example, cells of the immune system, such as T cells, may be isolated from the peripheral blood of a patient, using a commercially available cell separation system, such as CellPro Incorporated's (Bothell, Wash.) CEPRATE™ system (see U.S. Pat. No. 5,240,856; U.S. Pat. No. 5,215,926; WO 89/06280; WO 91/16116 and WO 92/07243). The separated cells are stimulated with one or more immunoreactive Mtb-81 polypeptides contained within a delivery vehicle, such as a microsphere, to provide antigen-specific T cells. The population of antigen-specific T cells is then expanded using standard techniques and the cells may be administered back to the patient as described, for example, by Chang et al., Crit. Rev. Oncol. Hematol. 22:213, 1996.

Within another embodiment, syngeneic or autologous dendritic cells may be pulsed with peptides corresponding to at least an immunogenic portion of Mtb-81 or Mtb-67.2. The resulting antigen-specific dendritic cells may either be transferred into a patient or employed to stimulate T cells to provide antigen-specific T cells which may, in turn, be administered to a patient. Alternatively, a vector expressing a polypeptide recited herein may be introduced into antigen presenting cells taken from a patient and clonally propagated ex vivo for transplant back into the same patient. Transfected cells may be reintroduced into the patient using any means known in the art, preferably in sterile form by intravenous, intracavitary or intraperitoneal administration.

Routes and frequency of administration, as well as dosage, will vary from individual to individual, and may be readily established using standard techniques. In general, the pharmaceutical compositions and vaccines may be administered by injection (e.g., intracutaneous, intramuscular, intravenous or subcutaneous), intranasally (e.g., by aspiration) or orally. Preferably, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of causing an immune response that leads to an improved clinical outcome (e.g., decreased symptoms or longer survival) in vaccinated patients as compared to non-vaccinated patients. In general, for pharmaceutical compositions and vaccines comprising one or more polypeptides, the amount of each polypeptide present in a dose ranges from about 100 μg to 5 mg per kg of host. Suitable dose sizes will vary with the size of the patient, but will typically range from about 0.1 mL to about 5 mL.

The following Examples are offered by way of illustration and not by way of limitation.

### EXAMPLES

### Example 1

### Preparation M. tuberculosis Protein

This Example illustrates the initial characterization of an M. tuberculosis protein that recognizes an antibody present in HIV positive individuals.

To identify M. tuberculosis antigens suitable for diagnostic methods, the high-molecular weight region of crude soluble proteins (CSP; obtained from Colorado State University) derived from M. tuberculosis strain H₃₇Rv was examined using two dimensional gel electrophoresis and two dimensional Western analysis. The probe for this analysis was monoclonal antibody IT57 (reviewed in Infection and Immunity 60:3925-3927, 1992), obtained from the UNPD/World Bank/World Health Organization Special Programme for Research and Training in Tropical Diseases. This antibody has been known to react with 82 kDa M. tuberculosis antigen(s) in this high-molecular weight region, but the identity of the protein antigen has not been previously elucidated (see Infection and Immunity 56:1994-1998, 1988; Infection and Immunity 60:3925-3927, 1992).

The CSP was separated by reverse phase chromatography on a C18 column. Approximately 75 mg of CSP was dissolved in water containing 0.1% trifluoroacetic acid (TFA), injected onto a C18 reverse phase column (22×250 mm, The Separations Group, Hesperia, Calif.) using a Prep LC (Waters, Milford, Mass.) and eluted with a binary gradient of 0.1% TFA in water (Solvent A) and acetonitrile (Solvent B) at a flow rate of 10 ml/minute. The gradient increased from 0 to 100% B in 60 min. Fractions were collected at 1 minute intervals.

Each fraction was individually tested by immunoblot analysis to identify the fractions containing reactivity against the IT57 antibody. Individual HPLC fractions were separated by SDS-PAGE on 4-20% gradient gels (BioRad, Hercules, Calif.) as per the manufacturers instructions prior to transfer to nitrocellulose. The proteins were transferred to nitrocellulose membranes (Hybond C Extra, Amersham, Arlington Heights, Ill.) and blocked with 0.5M NaCl in phosphate buffered saline (PBS) with 0.05% Tween (PBST). Blots were washed with PBS and probed with IT-57 antibody at a 1:50 or 1:70 dilution of culture supernatant in 0.5M NaCl in PBST for the 1D and 2D gels, respectively. After overnight incubation, blots were washed and probed with IgG specific donkey anti-mouse secondary antibody ECL (Jackson Immuno Research, West Grove, Pa.). Westerns were developed according to Pierce ECL protocol (Pierce Super Signal, Rockford, Ill.).

Fraction 38, which contained reactivity to antibody IT-57, was identified by Western analysis as described above and was further evaluated by 2D-PAGE and Western analysis. For 2D-PAGE analysis, Fraction 38 was concentrated to approximately 400 μl and 40 μl was added to 400 μl of rehydration solution containing 8 molar urea, 0.5% CHAPS (w/w), 15 mM DTT and 0.2% (w/v) Parmalyte pH 3-10. The solution was placed in a rehydration cassette and 18 cm pH 3-10 Immobiline Drystlips (Pharmacia Biotech, Uppsala, Sweden) were allowed to hydrate overnight. The hydrated strips were rinsed and focused using the multiphor II electrophoresis system with the Immobiline DryStrip kit and the EPS 3500 XL power supply from Pharmacia Biotech according to the following gradient: 0-300 volts/5minutes, 300-3500 volts/6 hours and 3500 thereafter to 80,000 volt/hours.

Tube gels for the ID control lanes were cast by adding 10 μl of each fraction to 10 μl of Tris acetate equilibration buffer from ESA (Chelmsford, Mass.) which contained 2% (w/v) DTT and 2% (w/v) agarose. The solution was heated at 100° C. for 5 minutes. Tube gels for molecular weight standards were cast by adding 2 μl of low range silver standards from Biorad (Hercules, Calif.) to 8 μl of water and 10 μl of Tris acetate equilibration buffer containing 2% (w/v) DTT and 2% (w/v) agarose and boiling for five minutes.

Focused Immobiline Drystrips were equilibrated in 10 mls/strip of equilibration buffer which contained 6 molar urea, 2% (w/v) SDS and 2% (w/v) DTT and rocked gently for 15 minutes. The buffer was decanted and the Drystrips were then equilibrated in 10 mls/strip of Tris acetate equilibration buffer which contained 6 molar urea and 2.5% iodoacetamide and rocked gently for 15 minutes. Strips were placed on top of Investigator 10% homogeneous double gels from ESA along side a 1 cm tube gel containing the same HPLC fraction as the DryStrip and a 1 cm tube gel containing low range silver standards from Biorad. The gels were run at 20 mA/gel overnight on the ESA Investigator 2D electrophoresis system which contained Tris acetate running buffer in the lower (anode) tank and Tris tricine SDS buffer in the upper (cathode) tank, both supplied by ESA.

One gel was transferred to nitrocellulose and immunoblotted using the IT57 antibody. The other gel was silver stained. For 2D-PAGE immunoblot analysis, the proteins were transferred to nitrocellulose membranes (Hybond C Extra, Amersham, Arlington Heights, Ill.) and blocked with 0.5M NaCl in phosphate buffered saline (PBS) with 0.05% Tween (PBST). Blots were washed with PBS and probed with IT-57 antibody at a 1:70 dilution of culture supernatant in 0.5M NaCl in PBST. After overnight incubation, blots were washed and probed with IgG specific donkey anti-mouse secondary antibody ECL (Jackson Immuno Research, West Grove, Pa.). Westerns were developed according to Pierce ECL protocol (Pierce Super Signal, Rockford, Ill.). For the silver stained gels, the gels were fixed for more than 1 hour and generally overnight in 40% methanol solution containing 10% acetic acid. The gels were then rinsed 3 times in 30% ethanol solution prior to reduction in 0.02% sodium thiosulfate in nanopure water for 1 minute. After reduction, the gels were washed 3 times in nanopure water for 20 seconds each wash before they were incubated for 20 minutes in 0.2% silver nitrate and 0.02% formaldehyde in nanopure water. The gels were washed 3 times for 20 seconds each wash before being developed in 3% sodium carbonate with 0.05% formaldehyde and 0.0005% sodium thiosulfate in nanopure water. After the gels were sufficiently developed the chemical process was stopped by the addition of 5% acetic acid. The gels were rinsed in nanopure water and stored in 0.1% acetic acid solution at 4° C.

The immunoblot analysis was correlated to the silver stained by staining the nitrocellulose membrane with AurodyeForte (Amersham Corp., Arlington Height, Ill.) total protein stain. Briefly, after developing immunobolots by ECL, the membranes were washed in PBS +0.3% Tween 3×5 min, rinsed in nanopure water, and then incubated in 40 ml AurodyeForte dye at room temp with gentle rocking until the desired proteins were visible.

The IT-57 reactive protein was excised from the gel and dehydrated by the addition of 100 μl of acetonitrile. The solvent was removed and replaced with 100 μl of 50 mM ammonium bicarbonate that contained 1 μl of 1M DTT, allowed to rehydrate, and then incubated at 57° C. for 1 hour. The solvent was removed, the gel dehydrated by the addition of 100 μl of acetonitrile. The solvent was replaced with 55 mM iodoacetamide in 50 mM ammonium bicarbonate after equilibration to room temperature and incubated in the dark at room temperature for 45 minutes with occasional vortexing. The gel was washed successively with 50 mM ammonium bicarbonate, acetonitrile, 50 mM ammonium bicarbonate, and acetonitrile before being fully dehydrated in a speedvac concentrator. Six μl of reductively alkylated trypsin, (Promega, Madison, Wis.) was added to 100 μl 50 mM ammonium bicarbonate the resulting suspension was incubated overnight at 37° C. The supernatant was removed and the tryptic peptides extracted with 1 wash of 100 mM ammonium bicarbonate followed by three successive washes of 50% acetonitrile with 15% formic acid. The extracts were pooled, concentrated on a speedvac to 30 μl volume, and stored at −20° C. until mass spectrometric analysis.

An aliquot of the tryptic peptides was loaded onto a C18 microcapillary column (75 μm i.d.×12 cm) and gradient eluted using acetonitrile and 0.1M acetic acid with the concentration of acetonitrile increasing from 0-80% in 12 minutes into a triple quadrupole mass spectrometer (TSQ7000; Finnigan MAT, San Jose, Calif.) equipped with an electrospray ionization source. Mass spectra were acquired every 1.5 seconds over a mass range of 300 to 1400 atomic mass units. Candidate peptide masses were identified by comparing the tryptic digest to a control digest.

To sequence the protein antigen, an addition aliquot of protein was generated by diphenyl fractionation. Approximately 5 mg of CSP was dissolved in water containing 0.5% trifluoroacetic acid (TFA). This sample was injected onto a Vydac diphenyl reverse phase column (Cat #219TP5415: The Separations Group, Hesperia, Calif.) eluted with a binary gradient of 0.5% TFA in water (buffer A) and acetonitrile (buffer B) on an AKTA explorer 100 separation system (Amersham Pharmacia Biotech AB, Uppsala, Sweden). The column was equilibrated in 30% B and a linear gradient was run from 30% to 65% buffer B at 2 ml/min over the course of 30 minutes. Fractions were collected at 1.5 ml intervals and analyzed by Western blotting using the IT57 antibody as described above. Fractions containing a protein recognized by this antibody eluted at about 50% B and were pooled and separated by SDS-PAGE. The gel was silver stained and digested in situ as described above.

Collision activated dissociation (CAD) mass spectra were recorded on the (M+2H)2+ ions at m/z 444 and 531. The CAD spectra were interpreted de novo or by peptide sequence tags (Anal Chem. 66(24):4390-99, 1994). The sequence for 444 2+ corresponded to the Cat G protein, the sequence for the 531 2+ corresponded to the Mtb-81 antigen. The M. tuberculosis genomic sequence used was from the published literature (Nature 393(6685) :537-44, 1998).

### Example 2

### Preparation of Mtb-67.2

This Example illustrates the identification and preparation of Mtb-67.2.

The high-molecular weight region of CSP derived from M. tuberculosis was examined using two dimensional gel electrophoresis. Five protein spots in the high molecular weight region were identified, individually excised, enzymatically digested and subjected to mass spectrometric analysis (as described in Example 1). The sequence of one of the identified proteins was determined and is provided herein as Mtb-81. Another protein, which appears to be present with Mtb-81 in a band that migrates in this high molecular weight region was found to be Mtb-67.2 (FIG. 5; SEQ ID NO:5).

### Example 3

### Preparation of Mtb-81 Polynucleotide

This Example illustrates the preparation of a DNA molecule encoding Mtb-81, and its expression product.

To obtain an Mtb-81 sequence for expression in E. coli, PCR analysis was performed using genomic M. tuberculosis DNA with the following primers (SEQ ID NOs: ______ and ______):

| | |
|---|---|
| PDM-268 | |
| 5′CTAAGTAGTACTGATCGCGTGTCGGTGGGC3′ | Tm=66° C. |
| PDM-269 | |
| 5′CAGTGAGAATTCACTAGCGGGCCGCATCGTCAC3′ | Tm=68° C. |

The PCR reactions contained:

10 μL 10× Pfu buffer

1 μL 10 mM dNTPs

2 μL each 10 μM oligonucleotide

83 μL sterile water

1.5 μL Pfu DNA polymerase (Stratagene)

50 ng M. tuberculosis genomic DNA

Reactions were heated to 96° C. for two minutes; cycled forty times at 96° C. (20 seconds), 67° C. (15 seconds), and 72° C. (5 minutes); and then incubated at 72° C. for 5 minutes. The PCR product was digested with Scal and EcoRI and cloned into pPDM His (a modified pET28 vector from Novagen, Madison, Wis.), which was digested with Eco72 I and Eco RI. Sequence was confirmed and the PCR product was transformed into BL21 pLys S (Novagen, Madison, Wis.). A single colony was inoculated into LB medium with kanamycin (30 μg/mL) and chloramphenicol (34 μg/mL). Twenty-four mL of the overnight culture was used to inoculate 1 liter of 2XYT broth with the same antibiotics in a baffled flask. Four liters were grown at once. At OD₅₆₀ of between 0.35 and 0.55, the flasks were induced with a final concentration of 1 mM IPTG. The bacteria were allowed to grow for four more hours before harvesting. The pellets were centrifuged and washed with 1×PBS and then centrifuged again. Pellets were resuspended in lysis buffer (20 mM Tris (pH 8.0). 100 mM NaCl and 0.1% DOC) and frozen at −20° C. overnight.

The pellets were then thawed and sonicated, and high speed centrifugation was used to separate the inclusion body pellet and the soluble supernatant. The Mtb-81 protein was found to be in the inclusion body pellet, and was washed twice with 0.5% CHAPS in 20 mM Tris (pH 8.0), 300 mM NaCl, and then solubilized in binding buffer (20 mM Tris, pH 8.0, 100 mM NaCl, 8M Urea). The pellet was then batch bound to Nickel NTA resin (Qiagen) and then passed over a Kontes (VWR) gravity flow column. The first wash was 20 mM Tris (pH 8.0), 350 mM NaCl, 1.0% DOC, 10 mM imidazole, 8M urea. The second wash was the same as the first, but without DOC. The elutions were done in a step wise manner, the first being 20 mM Tris (pH 8.0), 100 mM NaCl, 50 mM imidazole, 8 mM urea. The second increased the imidazole concentration to 100 mM. The third elution increased the imidazole to 500 mM. Less than one half the inclusion body did not stay bound to the Nickel and came off in the initial flowthrough. The protein started to elute with the lowest concentration of imidazole, and gradually came off the column as the imidazole concentration was increased. The elutions which contained the protein of interest were pooled and then dialyzed against 10 mM Tris (pH 8.0). After several dialysis changes, the protein was concentrated in a Vivaspin (IMS) 30 kD cutoff concentrator and then sterile filtered.

The results of whole protein composition analysis and predicted structural class (Protean application program within DNASTAR (Madison, Wis.) of the protein are presented in Tables I and II below.

TABLE I
Predicted Structural Class

| Analysis | Whole Protein |
|---|---|
| Molecular Weight | 81353.22 m.w. |
| Length | 748 |
| 1 microgram = | 12.292 pMoles |
| Molar Extinction coefficient | 77860 ± 5% |
| 1 A (280) = | 1.04 mg/ml |
| Isoelectric Point | 5.19 |
| Charge at pH 7 | −22.80 |

TABLE II
Whole Protein Composition Analysis

| | Number | % by | % by |
|---|---|---|---|
| Amino Acid(s) | count | weight | frequency |
| Charged (RKHYCDE) | 210 | 34.50 | 28.07 |
| Acidic (DE) | 98 | 14.48 | 13.10 |
| Basic (KR) | 72 | 12.93 | 9.63 |
| Polar (NCQSTY) | 158 | 21.64 | 21.12 |
| Hydrophobic (AILFWV) | 285 | 35.98 | 38.10 |
| A Ala | 87 | 7.60 | 11.63 |
| C Cys | 4 | 0.51 | 0.53 |
| D Asp | 62 | 8.77 | 8.29 |
| E Glu | 36 | 5.71 | 4.81 |
| F Phe | 23 | 4.16 | 3.07 |
| G Gly | 60 | 4.21 | 8.02 |
| H His | 20 | 3.37 | 2.67 |
| I Ile | 41 | 5.70 | 5.48 |
| K Lys | 26 | 4.10 | 3.48 |
| L Leu | 65 | 9.04 | 8.69 |
| M Met | 19 | 3.06 | 2.54 |
| N Asn | 29 | 4.07 | 3.88 |
| P Pro | 36 | 4.30 | 4.81 |
| Q Gin | 27 | 4.25 | 3.61 |
| R Arg | 46 | 8.83 | 6.15 |
| S Set | 34 | 3.64 | 4.55 |
| T Thr | 48 | 5.97 | 6.42 |
| V Val | 59 | 7.19 | 7.89 |
| W Trp | 10 | 2.29 | 1.34 |
| Y Tyr | 16 | 3.21 | 2.14 |
| B Asx | 0 | 0.00 | 0.00 |
| Z Glx | 0 | 0.00 | 0.00 |
| X Xxx | 0 | 0.00 | 0.00 |
| .Ter | 2 | 0.00 | 0.27 |

Expressed Mtb-81 was not recognized by murine monoclonal antibody IT-57 by Western analysis. This lack of reactivity may be due to limitations in the E. coli expression system (e.g., the protein may not be posttranslationally modified or may be improperly folded). Alternatively, another M. tuberculosis protein that reacts with IT-57 may remain to be identified. Mtb-81 protein was reactive against HIV-positive and M. tuberculosis-positive sera.

### Example 4

### Detection of Tuberculosis Using Mtb-81

This Example illustrates the use of Mtb-81 for serodiagnosis of M. tuberculosis infection in patients with and without HIV co-infection.

Reactivity of Mtb-81 was determined with sera from 47 normal (uninfected with M. tuberculosis) individuals, 27 patients that were HIV-positive and M. tuberculosis-positive, and 67 patients that were HIV-negative and M. tuberculosis-positive. Samples were defined as M. tuberculosis-positive if above the cutoff value, defined as the mean signal obtained from sera of normal individuals, plus three standard deviations.

ELISAs were performed in 96-well microtiter plates (Corning Easiwash), which were coated with Mtb-81 (200 ng/well). Coating was overnight at 4° C. Plates were then aspirated and blocked with phosphate buffered saline (PBS) containing 1% (w/v) BSA for two hours at room temperature, followed by a wash in PBS containing 0.1% Tween 20 (PBST). Serum (diluted 1/25 in PBST) was added to the wells and incubated for 30 minutes at room temperature. Following incubation, wells were washed six times with PBST and then incubated with Protein-A HRP conjugate at 1/20,000 dilution for 30 minutes. Plates were then washed six times in PBST and incubated with tetramethylbenzidine (TMB) substrate for a further 15 minutes. The reaction was stopped by the addition of 1 N sulfuric acid and plates were read at 450 nm using an ELISA plate reader. The cut-off for the assays was the mean of the negative population plus three standard deviations of the mean.

The results are presented in FIG. 2. Sera from 25 out of the 27 patients that were HIV-positive and M. tuberculosis-positive had an OD₄₅₀ above the cut-off value. None of the normal sera were above the cut-off, and 38 of the 67 serum samples from patients that were HIV-negative and M. tuberculosis-positive were above the cut-off value. These results demonstrate the use of Mtb-81 for serodiagnosis of M. tuberculosis infection.

### Example 5

### Preparation of Mtb-67.2 Polynucleotide

This Example illustrates the preparation of a DNA molecule encoding Mtb-67.2, and its expression product.

To obtain an Mtb-67.2 sequence for expression in E. coli, PCR analysis was performed using genomic M. tuberculosis DNA with the following primers (SEQ ID NOs:______ and ______):

| | |
|---|---|
| PEPCKHIS: | CAATTACATATGCATCACCATCACCATCACACCTCAGCGACCATCCCCGGTCTG |
| PEPCKTERM: | AAGATAAAGCTTCTAACCTAGGCGCTCCTTCAGG |

The PCR reactions contained:

10 μL 10× Pfu buffer

1 μL 10 mM dNTPs

2 μL each 10 μM oligonucleotide

83 μL sterile water

1.5 μL Pfu DNA polymerase (Stratagene)

50 ng M. tuberculosis genomic DNA

Reactions were heated to 94° C. for two minutes; cycled 35 times at 94° C. (30 seconds), 50° C. (145 seconds), and 72° C. (3 minutes); and then incubated at 72° C. for 5 minutes. The PCR product was digested with NdeI and HindIII and cloned into pET17b (Novagen; Madison, Wis.)), which was digested with NdeI and HindIII. Sequence was confirmed and the PCR product was transformed into BL21 pLys S (Novagen, Madison, Wis.). A single colony was inoculated into LB medium with ampicillin (100 μg/mL) and chloramphenicol (34 μg/mL). Twenty-four mL of the overnight culture was used to inoculate 1 liter of 2XYT broth with the same antibiotics in a baffled flask. Four liters were grown at once. At OD₅₆₀ of between 0.35 and 0.55, the flasks were induced with a final concentration of 1 mM IPTG. The bacteria were allowed to grow for four more hours before harvesting. The pellets were centrifuged and washed with 1× PBS and then centrifuged again. Pellets were resuspended in lysis buffer (20 mM Tris (pH 8.0). 100 mM NaCl and 0.1% DOC) and frozen at −20° C. overnight.

The pellets were then thawed and sonicated, and high speed centrifugation was used to separate the inclusion body pellet and the soluble supernatant. The Mtb-67.2 protein was found to be in the soluble supernatant, and was solubilized in binding buffer (20 mM Tris, pH 8.0, 100 mM NaCl, 8M Urea). The pellet was then batch bound to Nickel NTA resin (Qiagen) and then passed over a Kontes (VWR) gravity flow column. The first wash was 20 mM Tris (pH 8.0), 350 mM NaCl, 1.0% DOC, 10 mM imidazole, 8M urea. The second wash was the same as the first, but without DOC. The elutions were done in a step wise manner, the first being 20 mM Tris (pH 8.0), 100 mM NaCl, 50 mM imidazole, 8 mM urea. The second increased the imidazole concentration to 100 mM. The third elution increased the imidazole to 500 mM. The elutions which contained the protein of interest were pooled and then dialyzed against 10 mM Tris (pH 8.0). After several dialysis changes, the protein was sterile filtered.

The results of whole protein composition analysis and predicted structural class (Protean application program within DNASTAR (Madison, Wis.)) of the protein are presented in Tables III and IV below.

TABLE III
Predicted Structural Class

| Analysis | Whole Protein |
|---|---|
| Molecular Weight | 67252.27 m.w. |
| Length | 606 |
| 1 microgram = | 14.869 pMoles |
| Molar Extinction coefficient | 135360 ± 5% |
| 1 A (280) = | 0.50 ml/ml |
| Isoelectric Point | 4.83 |
| Charge at pH 7 | −25.28 |

TABLE IV
Whole Protein Composition Analysis

| Amino | Number | % by | % by |
|---|---|---|---|
| Acid(s) | count | weight | frequency |
| Charged (RKHYCDE) | 182 | 35.86 | 30.03 |
| Acidic (DE) | 86 | 15.61 | 14.19 |
| Basic (KR) | 59 | 12.54 | 9.74 |
| Polar (NCQSTY) | 123 | 20.44 | 20.30 |
| Hydrophobic (AILFWV) | 212 | 34.97 | 34.98 |
| A Ala | 54 | 5.71 | 8.91 |
| C Cys | 9 | 1.38 | 1.49 |
| D Asp | 43 | 7.36 | 7.10 |
| E Glu | 43 | 8.26 | 7.10 |
| F Phe | 26 | 5.69 | 4.29 |
| G Gly | 58 | 4.92 | 9.57 |
| H His | 12 | 2.45 | 1.98 |
| I Ile | 24 | 4.04 | 3.96 |
| K Lys | 28 | 5.34 | 4.62 |
| L Leu | 49 | 8.24 | 8.09 |
| M Met | 19 | 3.71 | 3.14 |
| N Asn | 22 | 3.73 | 3.63 |
| P Pro | 37 | 5.34 | 6.11 |
| Q Gln | 14 | 2.67 | 2.31 |
| R Arg | 31 | 7.20 | 5.12 |
| S Ser | 26 | 3.37 | 4.29 |
| T Thr | 36 | 5.41 | 5.94 |
| V Val | 39 | 5.75 | 6.44 |
| W Trp | 20 | 5.54 | 3.30 |
| Y Tyr | 16 | 3.88 | 2.64 |
| B Asx | 0 | 0.00 | 0.00 |
| Z Glx | 0 | 0.00 | 0.00 |
| X Xxx | 0 | 0.00 | 0.00 |
| .Ter | 0 | 0.00 | 0.00 |

Expressed Mtb-67.2 was not recognized by murine monoclonal antibody IT-57 by Western analysis. This lack of reactivity may be due to limitations in the E. coli expression system (e.g., the protein may not be posttranslationally modified or may be improperly folded). Alternatively, another M. tuberculosis protein that reacts with IT-57 may remain to be identified. Mtb-67.2 protein was reactive against HIV-positive and M. tuberculosis-positive sera.

### Example 6

### Detection of Tuberculosis Using Mtb-67.2

This Example illustrates the use of Mtb-67.2 for serodiagnosis of M. tuberculosis infection in patients with and without HIV co-infection.

Reactivity of Mtb-67.2 was determined with sera from 47 normal (uninfected with M. tuberculosis) individuals, 27 patients that were HIV-positive and M. tuberculosis-positive, and 67 patients that were HIV-negative and M. tuberculosis-positive. Samples were defined as M. tuberculosis-positive as described above.

ELISAs were performed in 96-well microtiter plates (Corning Easiwash), which were coated with Mtb-67.2 (200 ng/well). Coating was overnight at 4° C. Plates were then aspirated and blocked with phosphate buffered saline (PBS) containing 1% (w/v) BSA for two hours at room temperature, followed by a wash in PBS containing 0.1% Tween 20 (PBST). Serum (diluted 1/100 in PBST) was added to the wells and incubated for 30 minutes at room temperature. Following incubation, wells were washed six times with PBST and then incubated with Protein-A HRP conjugate at 1/20,000 dilution for 30 minutes. Plates were then washed six times in PBST and incubated with tetramethylbenzidine (TMB) substrate for a further 15 minutes. The reaction was stopped by the addition of 1 N sulfuric acid and plates were read at 450 nm using an ELISA plate reader. The cut-off for the assays was the mean of the negative population plus three standard deviations of the mean.

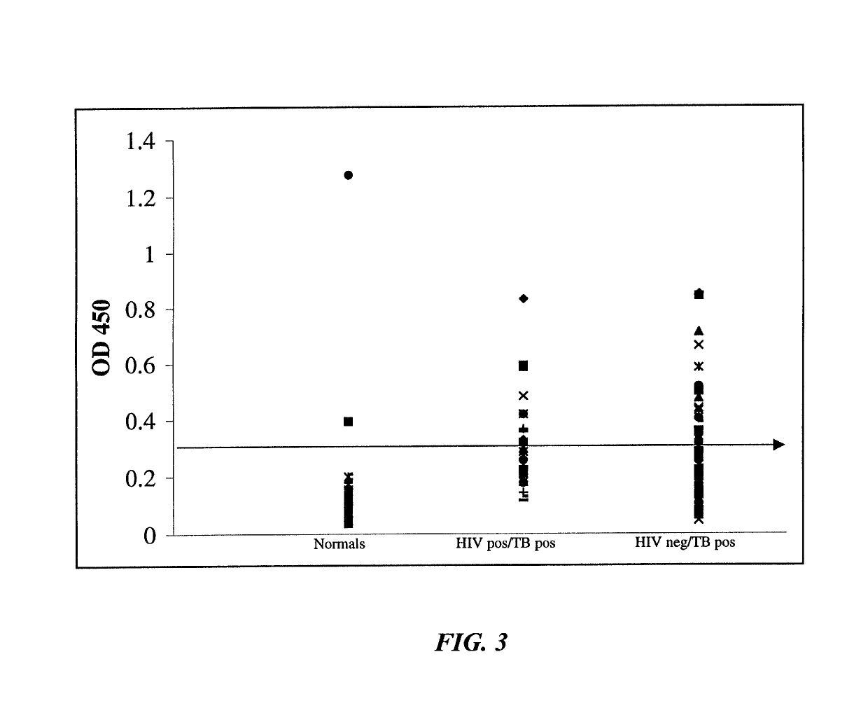

The results are presented in FIG. 3. Sera from 11 out of the 27 patients that were HIV-positive and M. tuberculosis-positive had an OD₄₅₀ above the cut-off value. Two out of 47 normal sera were above the cut-off, and 23 of the 67 serum samples from patients that were HIV-negative and M. tuberculosis-positive were above the cut-off value. These results demonstrate the use of Mtb-67.2 for serodiagnosis of M. tuberculosis infection.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. An isolated polypeptide comprising an immunogenic portion of Mtb-81 (FIGS. 1A-1F; SEQ ID NO:2), or a variant thereof that differs in one or more substitutions, additions, insertions and/or deletions such that the ability of the variant to react with Mtb-81-specific antisera or T-cells is not substantially diminished.

2. A polypeptide according to claim 1, wherein the polypeptide comprises at least nine consecutive amino acid residues of Mtb-81 (FIGS. 1A-1F; SEQ ID NO:2).

3. A polypeptide according to claim 1, wherein the polypeptide comprises at least 15 consecutive amino acid residues of Mtb-81 (FIGS. 1A-1F; SEQ ID NO:2).

4. A polypeptide according to claim 1, wherein the polypeptide comprises at least 50 consecutive amino acid residues of Mtb-81 (FIGS. 1A-1F; SEQ ID NO:2).

5. A polypeptide comprising an amino acid sequence recited in FIGS. 1A-1F (SEQ ID NO:2).

6. An isolated polynucleotide encoding a polypeptide according to claim 1.

7. A polynucleotide according to claim 6, wherein the polynucleotide comprises at least 15 consecutive nucleotides of the nucleotide sequence recited in FIGS. 1A-1F (SEQ ID NO: 1).

8. A polynucleotide according to claim 6, wherein the polynucleotide comprises at least 30 consecutive nucleotides of the nucleotide sequence recited in FIGS. 1A-1F (SEQ ID NO:1).

9. A polynucleotide comprising the nucleotide sequence recited in SEQ ID NO:1.

10. An expression vector comprising a polynucleotide according to claim 9.

11. A host cell transformed or transfected with an expression vector according to claim 10.

12. An antisense polynucleotide comprising at least 15 consecutive nucleotides complementary to the nucleotide sequence recited in FIGS. 1A-1F (SEQ ID NO:1).

13. An expression vector comprising a polynucleotide according to claim 12.

14. A host cell transformed or transfected with an expression vector according to claim 13.

15. A method for determining the presence or absence of M. tuberculosis in a biological sample, comprising the steps of:
(a) contacting a biological sample with:
(i) an isolated polypeptide according to claim 1; or
(ii) an antigen-presenting cell that expresses a polypeptide according to claim 1;
(b) detecting an amount of immunocomplexes formed between the polypeptide and antibodies in the biological sample that specifically bind to the polypeptide; and
(c) comparing the amount of immunocomplexes detected to a cut-off value, and therefrom determining the presence or absence of M. tuberculosis in the biological sample.

16. A method according to claim 15, wherein the polypeptide is linked to a solid support.

17. A method according to claim 16, wherein the support comprises nitrocellulose, latex or a plastic material.

18. A method according to claim 15, wherein the step of detecting comprises (a) incubating the immunocomplexes with a detection reagent that is capable of binding to the immunocomplexes, wherein the detection reagent comprises a reporter group, (b) removing unbound detection reagent, and (c) detecting the presence or absence of the reporter group.

19. A method according to claim 18, wherein the detection reagent comprises an antibody, or antigen-binding fragment thereof, capable of binding to antibodies that specifically bind to the polypeptide.

20. A method according to claim 18, wherein the reporter group is selected from the group consisting of radioisotopes, fluorescent groups, luminescent groups, enzymes, biotin, colloids and dye particles.

21. A method according to claim 15 wherein a reporter group is bound to the polypeptide, and wherein the step of detecting comprises removing unbound polypeptide and subsequently detecting the presence or absence of the reporter group.

22. A method according to claim 15, wherein the biological sample is selected from the group consisting of whole blood, serum, sputum, plasma, saliva, cerebrospinal fluid and urine.

23. A method for determining the presence or absence of M. tuberculosis infection in a patient, comprising the steps of:
(a) contacting a biological sample obtained from a patient with:
(i) an isolated polypeptide according to claim 1; or
(ii) an antigen-presenting cell that expresses a polypeptide according to claim 1;
(b) detecting an amount of immunocomplexes formed between the polypeptide and antibodies in the biological sample that specifically bind to the polypeptide; and
(c) comparing the amount of immunocomplexes detected to a cut-off value, and therefrom determining the presence or absence of M. tuberculosis infection in the patient.

24. A method according to claim 23, wherein the patient is infected with HIV.

25. A method for determining the presence or absence of M. tuberculosis infection in a patient, comprising the steps of:
(a) contacting a biological sample that comprises T cells and is obtained from a patient with an isolated polypeptide according to claim 1;
(b) detecting in the sample an amount of T cells that specifically react with the polypeptide; and
(c) comparing the amount of T cells detected to a cut-off value, and therefrom determining the presence or absence of M. tuberculosis in the patient.

26. A method according to claim 25, wherein the biological sample is selected from the group consisting of whole blood, serum, plasma and cerebrospinal fluid.

27. A method for determining the presence or absence of M. tuberculosis infection in a biological sample, comprising the steps of:
(a) detecting in a biological sample an amount of mRNA encoding a polypeptide according to claim 1; and
(b) comparing the amount of mRNA detected to a cut-off value, and therefrom determining the presence or absence of M. tuberculosis infection in the biological sample.

28. A method according to claim 27, wherein the step of detecting is performed using polymerase chain reaction.

29. A method according to claim 27, wherein the step of detecting is performed using a hybridization assay.

30. A method for determining the presence or absence of M. tuberculosis infection in a patient, comprising the steps of:
(a) detecting, in a biological sample obtained from a patient, an amount of mRNA encoding a polypeptide according to claim 1; and
(b) comparing the amount of mRNA detected to a cut-off value, and therefrom determining the presence or absence of M. tuberculosis infection in the patient.

31. A method according to claim 30, wherein the step of detecting is performed using polymerase chain reaction.

32. A method according to claim 30, wherein the step of detecting is performed using a hybridization assay.

33. A method for monitoring therapy in a patient infected by M. tuberculosis, the method comprising the steps of:
(a) contacting a biological sample obtained from a M. tuberculosis-infected patient at a first point in time with:
(i) an isolated polypeptide according to claim 1; or
(ii) an antigen-presenting cell that expresses a polypeptide according to claim 1;
(b) detecting an amount of immunocomplexes formed between the polypeptide and antibodies in the biological sample that specifically bind to the polypeptide;
(c) repeating steps (a) and (b) using a biological sample obtained at a second time point, wherein the second time point follows at least a portion of therapy for M. tuberculosis infection; and
(d) comparing the amount of immunocomplexes detected in step (a) with the amount detected in step (c), and therefrom monitoring the therapy for M. tuberculosis infection in the patient.

34. A method according to claim 33, wherein the patient is infected with HIV.

35. A method for monitoring therapy in a patient infected by M. tuberculosis, the method comprising the steps of:
(a) detecting, in a biological sample obtained from a M. tuberculosis-infected patient at a first point in time, an amount of mRNA encoding a polypeptide according to claim 1;
(b) detecting an amount of mRNA encoding a polypeptide according to claim 1 in a biological sample obtained from the patient at a second time point, wherein the second time point follows at least a portion of a therapy for M. tuberculosis infection; and
(c) comparing the amount of mRNA detected in step (a) to the amount detected in step (b), and therefrom monitoring the therapy for M. tuberculosis infection in the patient.

36. An isolated antibody, or antigen-binding fragment thereof, that specifically binds to Mtb-81 (SEQ ID NO:2).

37. An antibody according to claim 36, wherein the antibody is a monoclonal antibody.

38. A method for determining the presence or absence of M. tuberculosis in a biological sample, comprising the steps of:
(a) contacting a biological sample with an antibody or antigen-binding fragment thereof according to claim 36;
(b) detecting an amount of immunocomplexes formed between the antibody, or antigen-binding fragment thereof, and proteins in the biological sample that are specifically bound by the antibody, or antigen-binding fragment thereof; and
(c) comparing the amount of immunocomplexes detected to a cut-off value, and therefrom determining the presence or absence of M. tuberculosis in the biological sample.

39. A method according to claim 38, wherein the antibody, or antigen-binding fragment thereof, is linked to a solid support.

40. A method according to claim 39, wherein the support comprises nitrocellulose, latex or a plastic material.

41. A method according to claim 38, wherein the step of detecting comprises the steps of:
(a) incubating the immunocomplexes with a detection reagent that is capable of binding to the immunocomplexes, wherein the detection reagent comprises a reporter group;
(b) removing unbound detection reagent; and
(c) detecting the presence or absence of the reporter group.

42. A method according to claim 41, wherein the detection reagent comprises an antibody, or antigen-binding fragment thereof, capable of binding to the protein.

43. A method according to claim 41, wherein the reporter group is selected from the group consisting of radioisotopes, fluorescent groups, luminescent groups, enzymes, biotin, colloids and dye particles.

44. A method according to claim 38, wherein the step of detecting comprises the steps of:
(a) contacting the sample with an Mtb-81 polypeptide according to claim 1; and
(b) determining a level of inhibition of Mtb-81 polypeptide binding to the antibody or antigen-binding fragment thereof.

45. A method according to claim 44, wherein the Mtb-81 polypeptide comprises a reporter group.

46. A method according to claim 45, wherein the reporter group is selected from the group consisting of radioisotopes, fluorescent groups, luminescent groups, enzymes, biotin, colloids and dye particles.

47. A method according to claim 38, wherein the biological sample is selected from the group consisting of whole blood, serum, sputum, plasma, saliva, cerebrospinal fluid and urine.

48. A method for determining the presence or absence of M. tuberculosis infection in a patient, comprising the steps of:
(a) contacting a biological sample obtained from a patient with an antibody or antigen-binding fragment thereof according to claim 36;
(b) detecting an amount of immunocomplexes formed between the antibody, or antigen-binding fragment thereof, and proteins in the biological sample; and
(c) comparing the amount of immunocomplexes detected to a cut-off value, and therefrom determining the presence or absence of M. tuberculosis infection in the patient.

49. A method according to claim 48, wherein the biological sample is selected from the group consisting of whole blood, serum, plasma and cerebrospinal fluid.

50. A method for monitoring therapy in a patient infected by M tuberculosis, the method comprising the steps of:
(a) contacting a biological sample obtained from a M. tuberculosis-infected patient at a first time point with an antibody or antigen-binding fragment according to claim 36;
(b) detecting in the sample an amount of immunocomplexes formed between the antibody or antigen-binding fragment and proteins in the biological sample;
(c) repeating steps (a) and (b) using a biological sample obtained at a second time point, wherein the second time point follows at least a portion of therapy for M. tuberculosis infection; and
(d) comparing the amount of immunocomplexes detected in step (a) with the amount detected in step (c), and therefrom monitoring therapy in a patient infected by M. tuberculosis.

51. A diagnostic kit, comprising:
(a) a polypeptide according to claim 1; and
(b) a solid support.

52. A kit according to claim 51, wherein the polypeptide is immobilized on the solid support.

53. A kit according to claim 52, wherein the solid support comprises nitrocellulose, latex or a plastic material.

54. A diagnostic kit, comprising:
(a) a polypeptide according to claim 1; and
(b) a detection reagent.

55. A diagnostic kit, comprising:
(a) a polynucleotide according to claim 11; and
(b) a detection reagent.

56. A diagnostic kit, comprising:
(a) an antibody or antigen-binding fragment thereof according to claim 36; and
(b) an Mtb-81 polypeptide according to claim 1.

57. A fusion protein comprising a polypeptide according to claim I and a known M. tuberculosis antigen.

58. A pharmaceutical composition comprising:
(a) a polypeptide according to claim 1; and
(b) a physiologically acceptable carrier.

59. A vaccine comprising:
(a) a polypeptide according to claim 1; and
(b) a non-specific immune response enhancer.

60. A pharmaceutical composition comprising:
(a) a polynucleotide encoding a polypeptide according to claim 1; and
(b) a physiologically acceptable carrier.

61. A vaccine comprising:
(a) a polynucleotide encoding a polypeptide according to claim 1; and
(b) a non-specific immune response enhancer.

62. A pharmaceutical composition comprising:
(a) an antibody or antigen-binding fragment thereof that specifically binds to Mtb-81 (SEQ ID NO:2); and
(b) a physiologically acceptable carrier.

63. A pharmaceutical composition, comprising:
(a) an antigen presenting cell that expresses a polypeptide according to claim 1; and
(b) a physiologically acceptable carrier.

64. A pharmaceutical composition according to claim 63, wherein the antigen presenting cell is a dendritic cell or a macrophage.

65. A vaccine, comprising:
(a) an antigen presenting cell that expresses a polypeptide according to claim 1; and
(b) a non-specific immune response enhancer.

66. A vaccine according to claim 65, wherein the antigen presenting cell is a dendritic cell or a macrophage.

67. A method for inhibiting the development of tuberculosis in a patient, comprising administering to a patient an effective amount of a polypeptide according to claim 1, and thereby inhibiting the development of tuberculosis in the patient.

68. A method for inhibiting the development of tuberculosis in a patient, comprising administering to a patient an effective amount of a polynucleotide encoding a polypeptide according to claim 1, and thereby inhibiting the development of tuberculosis in the patient.

69. A method for inhibiting the development of tuberculosis in a patient, comprising administering to a patient an effective amount of an antibody or antigen-binding fragment thereof that specifically binds to Mtb-81 (SEQ ID NO:2), and thereby inhibiting the development of tuberculosis in the patient.

70. A method for inhibiting the development of tuberculosis in a patient, comprising administering to a patient an effective amount of an antigen presenting cell that expresses a polypeptide according to claim 1, and thereby inhibiting the development of tuberculosis in the patient.

71. A method according to claim 70, wherein the antigen presenting cell is a dendritic cell or a macrophage.

72. A method for stimulating and/or expanding T cells specific for Mtb-81, comprising contacting T cells with one or more of:
(i) a polypeptide according to claim 1;
(ii) a polynucleotide encoding such a polypeptide; and/or
(iii) an antigen presenting cell that expresses such a polypeptide;
under conditions and for a time sufficient to permit the stimulation and/or expansion of T cells.

73. An isolated T cell population, comprising T cells prepared according to the method of claim 72.

74. A method for inhibiting the development of tuberculosis in a patient, comprising administering to a patient a therapeutically effective amount of a T cell population according to claim 73.

75. A method for inhibiting the development of tuberculosis in a patient, comprising the steps of:
(a) incubating CD4⁺ and/or CD8+ T cells isolated from a patient with one or more of:
(i) a polypeptide according to claim 1;
(ii) a polynucleotide encoding such a polypeptide; or
(iii) an antigen-presenting cell that expresses such a polypeptide;
such that T cells proliferate; and
(b) administering to the patient an effective amount of the proliferated T cells, and thereby inhibiting the development of tuberculosis in the patient.

76. A method for inhibiting the development of tuberculosis in a patient, comprising the steps of:
(a) incubating CD4⁺ and/or CD8+ T cells isolated from a patient with one or more of:
(i) a polypeptide according to claim 1;
(ii) a polynucleotide encoding such a polypeptide; or
(iii) an antigen-presenting cell that expresses such a polypeptide;
such that T cells proliferate;
(b) cloning proliferated T cells; and
(c) administering to the patient an effective amount of the proliferated T cells, and thereby inhibiting the development of tuberculosis in the patient.

77. An isolated polypeptide comprising an immunogenic portion of Mtb-67.2 (FIG. 5; SEQ ID NO:5), or a variant thereof that differs in one or more substitutions, additions, insertions and/or deletions such that the ability of the variant to react with Mtb-67.2-specific antisera or T-cells is not substantially diminished.

78. A polypeptide according to claim 77, wherein the polypeptide comprises at least nine consecutive amino acid residues of Mtb-67.2 (FIG. 5; SEQ ID NO:5).

79. A polypeptide according to claim 77, wherein the polypeptide comprises at least 15 consecutive amino acid residues of Mtb-67.2 (FIG. 5; SEQ ID NO:5).

80. A polypeptide according to claim 77, wherein the polypeptide comprises at least 50 consecutive amino acid residues of Mtb-67.2 (FIG. 5; SEQ ID NO:5).

81. A polypeptide comprising the Mtb-67.2 sequence recited in FIG. 5 (SEQ ID NO:5).

82. An isolated polynucleotide encoding a polypeptide according to claim 77.

83. A polynucleotide according to claim 82, wherein the polynucleotide comprises at least 15 consecutive nucleotides of the Mtb-67.2 sequence recited in FIG. 4 (SEQ ID NO:4).

84. A polynucleotide comprising a nucleotide sequence recited in FIG. 4 (SEQ ID NO:4).

85. An expression vector comprising a polynucleotide according to claim 84.

86. A host cell transformed or transfected with an expression vector according to claim 85.

87. An antisense polynucleotide comprising at least 15 consecutive nucleotides complementary to the Mtb-67.2 sequence recited in FIG. 4 (SEQ ID NO:4).

88. An expression vector comprising a polynucleotide according to claim 87.

89. A host cell transformed or transfected with an expression vector according to claim 88.

90. A method for determining the presence or absence of M. tuberculosis in a biological sample, comprising the steps of:
(a) contacting a biological sample with:
(i) an isolated polypeptide according to claim 77; or
(ii) an antigen-presenting cell that expresses a polypeptide according to claim 77;
(b) detecting an amount of immunocomplexes formed between the polpeptide and antibodies in the biological sample that specifically bind to the polypeptide; and
(c) comparing the amount of immunocomplexes detected to a cut-off value, and therefrom determining the presence or absence of M. tuberculosis in the biological sample.

91. A method according to claim 90, wherein the polypeptide is linked to a solid support.

92. A method according to claim 91, wherein the support comprises nitrocellulose, latex or a plastic material.

93. A method according to claim 90, wherein the step of detecting comprises (a) incubating the immunocomplexes with a detection reagent that is capable of binding to the immunocomplexes, wherein the detection reagent comprises a reporter group, (b) removing unbound detection reagent, and (c) detecting the presence or absence of the reporter group.

94. A method according to claim 93, wherein the detection reagent comprises an antibody, or antigen-binding fragment thereof, capable of binding to antibodies that specifically bind to the polypeptide.

95. A method according to claim 93, wherein the reporter group is selected from the group consisting of radioisotopes, fluorescent groups, luminescent groups, enzymes, biotin, colloids and dye particles.

96. A method according to claim 90 wherein a reporter group is bound to the polypeptide, and wherein the step of detecting comprises removing unbound polypeptide and subsequently detecting the presence or absence of the reporter group.

97. A method according to claim 90, wherein the biological sample is selected from the group consisting of whole blood, serum, sputum, plasma, saliva, cerebrospinal fluid and urine.

98. A method for determining the presence or absence of M. tuberculosis infection in a patient, comprising the steps of:
(a) contacting a biological sample obtained from a patient with:
(i) an isolated polypeptide according to claim 77; or
(ii) an antigen-presenting cell that expresses a polypeptide according to claim 77;
(b) detecting an amount of immunocomplexes formed between the polypeptide and antibodies in the biological sample that specifically bind to the polypeptide; and
(c) comparing the amount of immunocomplexes detected to a cut-off value, and therefrom determining the presence or absence of M. tuberculosis infection in the patient.

99. A method according to claim 98, wherein the patient is infected with HIV.

100. A method for determining the presence or absence of M. tuberculosis infection in a patient, comprising the steps of:
(a) contacting a biological sample that comprises T cells and is obtained from a patient with an isolated polypeptide according to claim 77;
(b) detecting in the sample an amount of T cells that specifically react with the polypeptide; and
(c) comparing the amount of T cells detected to a cut-off value, and therefrom determining the presence or absence of M. tuberculosis in the patient.

101. A method according to claim 100, wherein the biological sample is selected from the group consisting of whole blood, serum, plasma and cerebrospinal fluid.

102. A method for determining the presence or absence of M. tuberculosis infection in a biological sample, comprising the steps of:
(a) detecting in a biological sample an amount of mRNA encoding a polypeptide according to claim 77; and
(b) comparing the amount of mRNA detected to a cut-off value, and therefrom determining the presence or absence of M. tuberculosis infection in the biological sample.

103. A method according to claim 102, wherein the step of detecting is performed using polymerase chain reaction.

104. A method according to claim 102, wherein the step of detecting is performed using a hybridization assay.

105. A method for determining the presence or absence of M. tuberculosis infection in a patient, comprising the steps of:
(a) detecting, in a biological sample obtained from a patient, an amount of mRNA encoding a polypeptide according to claim 77; and
(b) comparing the amount of mRNA detected to a cut-off value, and therefrom determining the presence or absence of M. tuberculosis infection in the patient.

106. A method according to claim 105, wherein the step of detecting is performed using polymerase chain reaction.

107. A method according to claim 105, wherein the step of detecting is performed using a hybridization assay.

108. A method for monitoring therapy in a patient infected by M tuberculosis, the method comprising the steps of:
(a) contacting a biological sample obtained from a M. tuberculosis-infected patient at a first point in time with:
(i) an isolated polypeptide according to claim 77; or
(ii) an antigen-presenting cell that expresses a polypeptide according to claim 77;
(b) detecting an amount of immunocomplexes formed between the polypeptide and antibodies in the biological sample that specifically bind to the polypeptide;
(c) repeating steps (a) and (b) using a biological sample obtained at a second time point, wherein the second time point follows at least a portion of therapy for M. tuberculosis infection; and
(d) comparing the amount of immunocomplexes detected in step (a) with the amount detected in step (c), and therefrom monitoring the therapy for M. tuberculosis infection in the patient.

109. A method according to claim 108, wherein the patient is infected with HIV.

110. A method for monitoring therapy in a patient infected by M. tuberculosis, the method comprising the steps of:
(a) detecting, in a biological sample obtained from a M. tuberculosis-infected patient at a first point in time, an amount of mRNA encoding a polypeptide according to claim 77;
(b) detecting an amount of mRNA encoding a polypeptide according to claim 77 in a biological sample obtained from the patient at a second time point, wherein the second time point follows at least a portion of a therapy for M. tuberculosis infection; and
(c) comparing the amount of mRNA detected in step (a) to the amount detected in step (b), and therefrom monitoring the therapy for M. tuberculosis infection in the patient.

111. An isolated antibody, or antigen-binding fragment thereof, that specifically binds to Mtb-67.2 (SEQ ID NO:5).

112. An antibody according to claim 111, wherein the antibody is a monoclonal antibody.

113. A method for determining the presence or absence of M. tuberculosis in a biological sample, comprising the steps of:
(a) contacting a biological sample with an antibody or antigen-binding fragment thereof according to claim 111;
(b) detecting an amount of immunocomplexes formed between the antibody, or antigen-binding fragment thereof, and proteins in the biological sample that are specifically bound by the antibody, or antigen-binding fragment thereof; and
(c) comparing the amount of immunocomplexes detected to a cut-off value, and therefrom determining the presence or absence of M. tuberculosis in the biological sample.

114. A method according to claim 113, wherein the antibody, or antigen-binding fragment thereof, is linked to a solid support.

115. A method according to claim 114, wherein the support comprises nitrocellulose, latex or a plastic material.

116. A method according to claim 113, wherein the step of detecting comprises the steps of:
(a) incubating the immunocomplexes with a detection reagent that is capable of binding to the immunocomplexes, wherein the detection reagent comprises a reporter group;
(b) removing unbound detection reagent; and
(c) detecting the presence or absence of the reporter group.

117. A method according to claim 116, wherein the detection reagent comprises an antibody, or antigen-binding fragment thereof, capable of binding to the protein.

118. A method according to claim 116, wherein the reporter group is selected from the group consisting of radioisotopes, fluorescent groups, luminescent groups, enzymes, biotin, colloids and dye particles.

119. A method according to claim 113, wherein the step of detecting comprises the steps of:
(a) contacting the sample with an Mtb-67.2 polypeptide according to claim 77; and
(b) determining a level of inhibition of Mtb-67.2 polypeptide binding to the antibody or antigen-binding fragment thereof.

120. A method according to claim 119, wherein the Mtb-67.2 polypeptide comprises a reporter group.

121. A method according to claim 120, wherein the reporter group is selected from the group consisting of radioisotopes, fluorescent groups, luminescent groups, enzymes, biotin, colloids and dye particles.

122. A method according to claim 113, wherein the biological sample is selected from the group consisting of whole blood, serum, sputum, plasma, saliva, cerebrospinal fluid and urine.

123. A method for determining the presence or absence of M. tuberculosis infection in a patient, comprising the steps of:
(a) contacting a biological sample obtained from a patient with an antibody or antigen-binding fragment thereof according to claim 111;
(b) detecting an amount of immunocomplexes formed between the antibody, or antigen-binding fragment thereof, and proteins in the biological sample; and
(c) comparing the amount of immunocomplexes detected to a cut-off value, and therefrom determining the presence or absence of M. tuberculosis infection in the patient.

124. A method according to claim 123, wherein the biological sample is selected from the group consisting of whole blood, serum, plasma and cerebrospinal fluid.

125. A method for monitoring therapy in a patient infected by M. tuberculosis, the method comprising the steps of:
(a) contacting a biological sample obtained from a M. tuberculosis-infected patient at a first time point with an antibody or antigen-binding fragment according to claim 111;
(b) detecting in the sample an amount of immunocomplexes formed between the antibody or antigen-binding fragment and proteins in the biological sample;
(c) repeating steps (a) and (b) using a biological sample obtained at a second time point, wherein the second time point follows at least a portion of therapy for M. tuberculosis infection; and
(d) comparing the amount of immunocomplexes detected in step (a) with the amount detected in step (c), and therefrom monitoring therapy in a patient infected by M. tuberculosis.

126. A diagnostic kit, comprising:
(a) a polypeptide according to claim 77; and
(b) a solid support.

127. A kit according to claim 126, wherein the polypeptide is immobilized on the solid support.

128. A kit according to claim 127, wherein the solid support comprises nitrocellulose, latex or a plastic material.

129. A diagnostic kit, comprising:
(a) a polypeptide according to claim 77; and
(b) a detection reagent.

130. A diagnostic kit, comprising:
(a) a polynucleotide according to claim 87; and
(b) a detection reagent.

131. A diagnostic kit, comprising:
(a) an antibody or antigen-binding fragment thereof according to claim 111; and
(b) an Mtb-67.2 polypeptide according to claim 77.

132. A fusion protein comprising a polypeptide according to claim 77 and a known M. tuberculosis antigen.

133. A pharmaceutical composition comprising:
(a) a polypeptide according to claim 77; and
(b) a physiologically acceptable carrier.

134. A vaccine comprising:
(a) a polypeptide according to claim 77; and
(b) a non-specific immune response enhancer.

135. A pharmaceutical composition comprising:
(a) a polynucleotide encoding a polypeptide according to claim 77; and
(b) a physiologically acceptable carrier.

136. A vaccine comprising:
(a) a polynucleotide encoding a polypeptide according to claim 77; and
(b) a non-specific immune response enhancer.

137. A pharmaceutical composition comprising:
(a) an antibody or antigen-binding fragment thereof that specifically binds to Mtb-67.2 (SEQ ID NO:5); and
(b) a physiologically acceptable carrier.

138. A pharmaceutical composition, comprising:
(a) an antigen presenting cell that expresses a polypeptide according to claim 77; and
(b) a physiologically acceptable carrier.

139. A pharmaceutical composition according to claim 138, wherein the antigen presenting cell is a dendritic cell or a macrophage.

140. A vaccine, comprising:
(a) an antigen presenting cell that expresses a polypeptide according to claim 77; and
(b) a non-specific immune response enhancer.

141. A vaccine according to claim 140, wherein the antigen presenting cell is a dendritic cell or a macrophage.

142. A method for inhibiting the development of tuberculosis in a patient, comprising administering to a patient an effective amount of a polypeptide according to claim 77, and thereby inhibiting the development of tuberculosis in the patient.

143. A method for inhibiting the development of tuberculosis in a patient, comprising administering to a patient an effective amount of a polynucleotide encoding a polypeptide according to claim 77, and thereby inhibiting the development of tuberculosis in the patient.

144. A method for inhibiting the development of tuberculosis in a patient, comprising administering to a patient an effective amount of an antibody or antigen-binding fragment thereof that specifically binds to Mtb-67.2 (SEQ ID NO:5), and thereby inhibiting the development of tuberculosis in the patient.

145. A method for inhibiting the development of tuberculosis in a patient, comprising administering to a patient an effective amount of an antigen presenting cell that expresses a polypeptide according to claim 77, and thereby inhibiting the development of tuberculosis in the patient.

146. A method according to claim 145, wherein the antigen presenting cell is a dendritic cell or a macrophage.

147. A method for stimulating and/or expanding T cells specific for Mtb-67.2, comprising contacting T cells with one or more of:
(i) a polypeptide according to claim 77;
(ii) a polynucleotide encoding such a polypeptide; and/or
(iii) an antigen presenting cell that expresses such a polypeptide;
under conditions and for a time sufficient to permit the stimulation and/or expansion of T cells.

148. An isolated T cell population, comprising T cells prepared according to the method of claim 147.

149. A method for inhibiting the development of tuberculosis in a patient, comprising administering to a patient an effective amount of a T cell population according to claim 148.

150. A method for inhibiting the development of tuberculosis in a patient, comprising the steps of:
(a) incubating CD4⁺ and/or CD8+ T cells isolated from a patient with one or more of:
(i) a polypeptide according to claim 77;
(ii) a polynucleotide encoding such a polypeptide; or
(iii) an antigen-presenting cell that expresses such a polypeptide;
such that T cells proliferate; and
(b) administering to the patient an effective amount of the proliferated T cells, and thereby inhibiting the development of tuberculosis in the patient.

151. A method for inhibiting the development of tuberculosis in a patient, comprising the steps of:
(a) incubating CD4⁺ and/or CD8+ T cells isolated from a patient with one or more of:
(i) a polypeptide according to claim 77;
(ii) a polynucleotide encoding such a polypeptide; or
(iii) an antigen-presenting cell that expresses such a polypeptide;
such that T cells proliferate;
(b) cloning proliferated T cells; and
(c) administering to the patient an effective amount of the proliferated T cells, and thereby inhibiting the development of tuberculosis in the patient.
